(19) United States
(12) Patent Application Publication
(10) Pub. No.: US 2016/0333418 A1
(43) Pub. Date: Nov. 17, 2016

(54) Activin Inhibitor Response Prediction and Uses for Treatment

(71) Applicant: SANTA MARIA BIOTHERAPEUTICS, INC., South San Francisco, CA (US)

(72) Inventor: Christopher Michael Haqq, Newbury Park, CA (US)

(21) Appl. No.: 15/111,034

(22) PCT Filed: Jan. 14, 2015
(86) PCT No.: PCT/US15/11396

§ 371 Date: Jul. 12, 2016

Related U.S. Application Data
(60) Provisional application No. 61/927,413, filed on Jan. 14, 2014.
(60) Provisional application No. 62/022,842, filed on Jul. 10, 2014.

(51) Int. Cl. C12Q 1/68; A61K 38/17

(52) U.S. Cl. CPC C12Q 1/6886; A61K 38/179; C12Q 2600/158; C12Q 2600/106

(57) ABSTRACT

Described herein are methods and assays for determining the presence or absence of certain markers useful in informing treatment of one or more subjects with one or more activin inhibitors. In certain aspects the one or more subjects can have cancer. In certain aspects the markers can include INHBA and ACVR2B. In certain aspects one or more subjects can be treated with an activin inhibitor.

### CROSS REFERENCE TO RELATED APPLICATIONS

This application claims priority to U.S. Provisional Application No. 61/927,413, filed Jan. 14, 2014, and U.S. Provisional Application No. 62/022,842, filed Jul. 10, 2014; the entire disclosures of which are relied upon and incorporated by reference herein, in their entirety, for all purposes.

### REFERENCE TO THE SEQUENCE LISTING

The present application is being filed along with a Sequence Listing in electronic format. The Sequence Listing is provided as a file entitled XXXXX.txt, created XXXX, which is XX KB in size. The information in the electronic format of the Sequence Listing is incorporated herein by reference in its entirety.

### BACKGROUND

The transforming growth factor β (TGF-β) family of proteins includes the transforming growth factors-β (TGF-β), activins, bone morphogenic proteins (BMP), nerve growth factors (NGFs), brain-derived neurotrophic factor (BDNF), and growth/differentiation factors (GDFs). These family members are involved in the regulation of a wide range of biological processes including cell proliferation, differentiation, and other functions.

Growth/differentiation factor 8 (GDF-8), also referred to as myostatin, is a TGF-β family member expressed for the most part in the cells of developing and adult skeletal muscle tissue. Myostatin appears to play an essential role in negatively controlling skeletal muscle growth (McPherson et al., Nature (London) 387, 83-90 (1997), Zimmers et al., Science 296:1486-1488 (2002)). Antagonizing myostatin has been shown to increase lean muscle mass in animals.

Another member of the TGF-β family of proteins is a related growth/differentiation factor, growth/differentiation factor 11 (GDF-11). GDF-11 has approximately 90% sequence identity to the amino acid sequence of myostatin. GDF-11 has a role in the axial patterning in developing animals (Oh et al., Genes Dev 11:1812-26 (1997)), and also appears to play a role in skeletal muscle development and growth.

Activins A, B and AB are the homodimers and heterdimer respectively of two polypeptide chains, βA and βB (Vale et al., Nature 321, 776-779 (1986), Ling et al., Nature 321, 779-782 (1986)). Activins were originally discovered as gonadal peptides involved in the regulation of follicle stimulating hormone synthesis, and are now believed to be involved in the regulation of a number of biological activities. Activin A is a predominant form of activin.

Activin, myostatin, GDF-11 and other members of the TGF-β superfamily bind and signal through a combination of activin type II and activin type IIB receptors, both of which are transmembrane serine/threonine kinases (Harrison et al., J. Biol. Chem. 279, 28036-28044 (2004)). Cross-linking studies have determined that myostatin is capable of binding the activin type II receptors ActRIIA and ActRIIB in vitro (Lee et al., PNAS USA 98:9306-11 (2001)). There is also evidence that GDF-11 binds to both ActRIIA and ActRIIB (Oh et al., Genes Dev 16:2749-54 (2002)).

TGF-β protein expression is known to be associated with a variety of diseases and disorders. Therefore, therapeutic molecules capable of antagonizing several TGF-β proteins simultaneously may be particularly effective for treating these diseases and disorders.

This application is related to U.S. Pat. No. 8,410,043, filed Nov. 25, 2009; and U.S. Pat. No. 7,947,646, filed Mar. 5, 2008, the entire disclosures of which are relied upon and incorporated by reference herein, in their entirety, for all purposes.

### SUMMARY

Disclosed herein is a method for treating a subject with an activin inhibitor, comprising: performing an assay on a sample from the subject to generate a dataset comprising data representing the expression of at least two markers comprising inhibin beta A (INHBA) and activin A receptor type IIB (ACVR2B); determining, based on the dataset, the likelihood that the subject will respond to treatment with the activin inhibitor, wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; and administering the activin inhibitor to the subject if there is detectable expression of INHBA and ACVR2B within the sample.

In some aspects, the assay is an in situ hybridization assay performed using a plurality of distinct probes, wherein the subject has cancer, wherein the sample comprises one or more cancer cells, wherein the cancer cells are ovary, endometrial, pancreas, bile duct, lung, gastric, head/neck, breast, colorectal, melanoma, or testicular cancer cells, and wherein the activin inhibitor is a polypeptide comprising the amino acid sequence set forth in SEQ ID NO:10.

In some aspects, the assay is a nucleotide-based assay, optionally wherein the nucleotide-based assay is an in situ hybridization assay performed using a plurality of distinct probes, optionally wherein the plurality of distinct probes hybridize to the nucleotides located at positions 364-1374 of the nucleotide sequence shown in SEQ ID NO:51 or the nucleotides located at positions 627-1503 of the nucleotide sequence shown in SEQ ID NO:52.

In some aspects, the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

In some aspects, the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

In some aspects, the subject is a human subject. In some aspects, the subject has cancer.

In some aspects, the dataset further comprises data representing the location of the at least two markers relative to each other, optionally wherein co-localization of the markers indicates that the subject is more likely to be responsive to treatment with the activin inhibitor, optionally wherein the co-localization of the markers is in a single cell indicating autocrine signaling, and optionally wherein the co-localization of the markers is in neighboring cells indicating paracrine signaling.

Also disclosed herein is a method for determining the likelihood that a subject will respond to treatment with an activin inhibitor, comprising: performing an assay on a sample from the subject to generate a dataset comprising data representing the expression of at least two markers comprising INHBA and ACVR2B; and determining, based on the dataset, the likelihood that the subject will respond to treatment with the activin inhibitor, wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment.

In some aspects, the assay is an in situ hybridization assay performed using a plurality of distinct probes, wherein the subject has cancer, wherein the sample comprises RNA from one or more cancer cells, wherein the cancer cells are ovary, endometrial, pancreas, bile duct, lung, gastric, head/neck, breast, colorectal, melanoma, or testicular cancer cells, and wherein the activin inhibitor is a polypeptide comprising the amino acid sequence set forth in SEQ ID NO:10.

In some aspects, the assay is a nucleotide-based assay, optionally wherein the nucleotide-based assay is an in situ hybridization assay performed using a plurality of distinct probes, optionally wherein the plurality of distinct probes hybridize to the nucleotides located at positions 364-1374 of the nucleotide sequence shown in SEQ ID NO:51 or the nucleotides located at positions 627-1503 of the nucleotide sequence shown in SEQ ID NO:52.

In some aspects, the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

In some aspects, the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

In some aspects, the subject is a human subject. In some aspects, the subject has cancer. In some aspects, the method further comprises administering the activin inhibitor to the subject.

In some aspects, the dataset further comprises data representing the location of the at least two markers relative to each other, optionally wherein co-localization of the markers indicates that the subject is more likely to be responsive to treatment with the activin inhibitor, optionally wherein the co-localization of the markers is in a single cell indicating autocrine signaling, and optionally wherein the co-localization of the markers is in neighboring cells indicating paracrine signaling.

Also disclosed herein is a method for assaying the expression of INHBA and ACVR2B in a sample from a subject, comprising: performing an in situ hybridization assay on the sample using a plurality of distinct probes that hybridize to the nucleotides located at positions 364-1374 of the nucleotide sequence shown in SEQ ID NO:51 or the nucleotides located at positions 627-1503 of the nucleotide sequence shown in SEQ ID NO:52; and determining the expression of INHBA and ACVR2B based on the assay, wherein detectable expression of INHBA and ACVR2B within the sample indicates that the subject is more likely to be responsive to treatment with an activin inhibitor, and/or wherein the lack of detectable expression of at least one of INHBA and ACVR2B within the sample indicates that the subject is less likely to be responsive to treatment with the activin inhibitor.

In some aspects, the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

In some aspects, the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

In some aspects, the subject is a human subject. In some aspects, the subject has cancer. In some aspects, the method further comprises administering the activin inhibitor to the subject.

In some aspects, co-localization of the markers indicates that the subject is more likely to be responsive to treatment with the activin inhibitor, optionally wherein the co-localization of the markers is in a single cell indicating autocrine signaling, and optionally wherein the co-localization of the markers is in neighboring cells indicating paracrine signaling.

Also disclosed herein is a method for determining the likelihood that a subject will respond to activin inhibitor therapy, comprising: obtaining a dataset obtained from a sample from the subject, wherein the dataset comprises data representing the expression of at least two markers comprising INHBA and ACVR2B and, optionally, data representing the relative location of each of the markers within the sample; and determining, based on the dataset, the likelihood that the subject will respond to activin inhibitor therapy, wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; or wherein higher expression of at least one of INHBA and ACVR2B relative to a control in combination with co-localization of INHBA and ACVR2B within the sample indicates that the subject is more likely to be responsive to activin inhibitor therapy than a second subject lacking at least one of higher expression of at least one of INHBA and ACVR2B relative to a control and co-localization of INHBA and ACVR2B within the sample.

In some aspects, the method is implemented on a computer. In some aspects, obtaining the dataset obtained from the sample comprises obtaining the sample and processing the sample to experimentally determine the dataset; or wherein obtaining the dataset obtained from the sample comprises receiving the dataset from a third party that has processed the sample to experimentally determine the dataset. In some aspects, the sample comprises RNA from a cancer cell. In some aspects, the data are hybridization data. In some aspects, the dataset is obtained stored on a storage memory.

In some aspects, the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

In some aspects, the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

In some aspects, the subject is a human subject. In some aspects, the subject has cancer. In some aspects, the method further comprises administering the activin inhibitor to the subject.

Also disclosed herein is a method of treating a subject with an activin inhibitor, comprising: obtaining a dataset obtained from a sample from the subject, wherein the dataset comprises data representing the expression of at least two markers comprising INHBA and ACVR2B and, optionally, data representing the relative location of each of the markers within the sample; determining, based on the dataset, the likelihood that the subject will respond to activin inhibitor therapy, wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; or wherein higher expression of at least one of INHBA and ACVR2B relative to a control in combination with co-localization of INHBA and ACVR2B within the sample indicates that the subject is more likely to be responsive to activin inhibitor therapy than a second subject lacking at least one of higher expression of at least one of INHBA and ACVR2B relative to a control and co-localization of INHBA and ACVR2B within the sample; and administering the activin inhibitor to the subject.

In some aspects, obtaining the dataset obtained from the sample comprises obtaining the sample and processing the sample to experimentally determine the dataset; or wherein obtaining the dataset obtained from the sample comprises receiving the dataset from a third party that has processed the sample to experimentally determine the dataset. In some aspects, the sample comprises RNA from a cancer cell. In some aspects, the data are hybridization data. In some aspects, the dataset is obtained stored on a storage memory.

In some aspects, the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

In some aspects, the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

In some aspects, the subject is a human subject. In some aspects, the subject has cancer.

Also disclosed herein is a system for determining the likelihood that a subject will respond to activin inhibitor therapy, the system comprising: a storage memory for storing a dataset obtained from a sample from the subject, wherein the dataset comprises data representing the expression of at least two markers comprising INHBA and ACVR2B and, optionally, data representing the relative location of each of the markers within the sample;

and a processor communicatively coupled to the storage memory for determining, based on the dataset, the likelihood that the subject will respond to activin inhibitor therapy, wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; or wherein higher expression of at least one of INHBA and ACVR2B relative to a control in combination with co-localization of INHBA and ACVR2B within the sample indicates that the subject is more likely to be responsive to activin inhibitor therapy than a second subject lacking at least one of higher expression of at least one of INHBA and ACVR2B relative to a control and co-localization of INHBA and ACVR2B within the sample.

In some aspects, disclosed herein is a system for implementation of a method disclosed herein or a portion of a method disclosed herein.

Also disclosed herein is a computer-readable storage medium storing computer-executable program code for scoring a sample obtained from a subject, the medium comprising: a dataset obtained from a sample from the subject, wherein the dataset comprises data representing the expression of at least two markers comprising INHBA and ACVR2B and, optionally, data representing the relative location of each of the markers within the sample; and computer-executable program code for determining, based on the dataset, the likelihood that the subject will respond to activin inhibitor therapy, wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; or wherein higher expression of at least one of INHBA and ACVR2B relative to a control in combination with co-localization of INHBA and ACVR2B within the sample indicates that the subject is more likely to be responsive to activin inhibitor therapy than a second subject lacking at least one of higher expression of at least one of INHBA and ACVR2B relative to a control and co-localization of INHBA and ACVR2B within the sample.

In some aspects, disclosed herein is a computer readable storage medium for implementation of a method disclosed herein or a portion of a method disclosed herein.

### BRIEF DESCRIPTION OF THE FIGURES

FIG. 1 shows a comparison between ActRIIB-Fc (E28W) and svActRIIB-Fc (E28W, S44T) on an SEC column. svActRIIB-Fc (E28W, S44T) shows a single peak compared with ActRIIB-Fc (E28W), which shows three peaks.

FIG. 2 shows the increase in body mass over a 14 day period in 10 C57B1/6 mice administered a single dose of 10 mg/kg svActRIIB-Fc (E28W, S44T) compared with 10 mice administered 10 mg/kg of PBS.

FIG. 3 shows the dose-related change in lean body mass over time for C57B1/6 receiving a single dose of 0.3 mg/kg, 3 mg/kg, 10 mg/kg, and 30 mg/kg of svActRIIB-Fc (E28W, S44T).

FIG. 4 shows an overview of a typical RNAscope® technology workflow.

FIG. 5 shows an example of each scoring criteria (0, 1, 2, 3, and 4) using RNA in situ hybridization via RNAscope®.

### DETAILED DESCRIPTION

The present invention provides an isolated protein comprising a stabilized human activin IIB receptor (svActRIIB) polypeptide. The protein and polypeptide of the invention are characterized by their ability to bind to at least one of three TGF-β proteins, myostatin (GDF-8), activin A, or GDF-11, to inhibit the activities of at least one of these proteins, and to have improved manufacturability properties compared with other ActRIIB soluble receptors. The stabilized human activin IIB receptor polypeptide is characterized by amino acid substitutions at both positions E28 and S44 with reference to the extracellular domain of ActRIIB, as set forth in SEQ ID NO: 2. In one embodiment, a stabilized human activin IIB receptor polypeptide can have a further substitution of alanine at position 64 with respect to SEQ ID NO: 2.

As used herein the term “TGF-β family members” or “TGF-β proteins” refers to the structurally related growth factors of the transforming growth factor family including activins, and growth and differentiation factor (GDF) proteins (Kingsley et al. Genes Dev. 8: 133-146 (1994), McPherron et al., Growth factors and cytokines in health and disease, Vol. 1B, D. LeRoith and C. Bondy. ed., JAI Press Inc., Greenwich, Conn., USA: pp 357-393).

GDF-8, also referred to as myostatin, is a negative regulator of skeletal muscle tissue (McPherron et al. PNAS USA 94:12457-12461 (1997)). Myostatin is synthesized as an inactive protein approximately 375 amino acids in length, having GenBank Accession No: AAB86694 (SEQ ID NO: 35) for human, as well as variants and species homologs of that protein. The precursor protein is activated by proteolytic cleavage at a tetrabasic processing site to produce an N-terminal inactive prodomain and an approximately 109 amino acid C-terminal protein which dimerizes to form a homodimer of about 25 kDa. This homodimer is the mature, biologically active protein (Zimmers et al., Science 296, 1486 (2002)).

As used herein, the term “prodomain” or “propeptide” refers to the inactive N-terminal protein which is cleaved off to release the active C-terminal protein. As used herein the term “myostatin” or “mature myostatin” refers to the mature, biologically active C-terminal polypeptide, in monomer, dimer or other form, as well as biologically active fragments or related polypeptides including allelic variants, splice variants, and fusion peptides and polypeptides. The mature myostatin has been reported to have 100% sequence identity among many species including human, mouse, chicken, porcine, turkey, and rat (Lee et al., PNAS 98, 9306 (2001)).

As used herein GDF-11 refers to the BMP (bone morphogenic protein) having Swissprot accession number 095390 (SEQ ID NO: 36), as well as variants and species homologs of that protein. GDF-11 is involved in the regulation of anterior/posterior patterning of the axial skeleton (McPherson et al, Nature Genet. 22 (93): 260-264 (1999); Gamer et al, Dev. Biol. 208 (1), 222-232 (1999)) but postnatal functions are unknown.

Activin A is the homodimer of the polypeptide chains βA. The term “activin A” can refer to the activin protein having GenBank Accession No: NM_002192 (SEQ ID NO: 34), as well as variants and species homologs of that protein. Activins A, B, and AB are the homodimers and heterodimer respectively of two polypeptide chains, βA and βB. As used herein, “activin” refers to activin A, B, and AB, as well as variants and species homologs of that protein.

The terms “anti-activin compound”, “activin inhibitor” and “activin antagonist” are used interchangeably. Each is a molecule that binds to and detectably inhibits at least one function of activin-A via an assay. Conversely, an “activin agonist” is a molecule that detectably increases at least one function of activin-A. The inhibition caused by an activin inhibitor need not be complete so long as it is detectable using an assay. Any assay of a function of activin-A can be used, examples of which are provided herein. Examples of functions of activin-A that can be inhibited by an activin inhibitor, or increased by an activin agonist, include binding to activin-A. Examples of types of activin inhibitors and activin agonists include, but are not limited to, activin binding polypeptides such as antigen binding proteins (e.g., activin-A inhibiting antigen binding proteins), stabilized variant activin IIB receptors (svActRIIB), svActRIIB fragments, svActRIIB derivatives, antibodies, antibody fragments, and antibody derivatives. In addition, examples of activin inhibitors include those disclosed herein (e.g., variant ActRIIBs such as svActRIIB-based polypeptides), those disclosed in PCT/US2014/14490, those disclosed in PCT/US2012/070571, those disclosed in U.S. Ser. No. 14/085,056, those disclosed in U.S. Pat. No. 7,947,646, those disclosed in U.S. Pat. Nos. 8,486,403, 8,361,957, 8,703,927, 8,343,933, and 8,252,900, and those disclosed in U.S. Ser. Nos. 13/796,135, 13/404,593, 13/403,657, 13/750,249, 13/730,418, and 13/588,468; each of which is herein incorporated by reference, in its entirety, for all purposes.

A “subject” in the context of the present teachings is generally a mammal The subject can be a human patient, e.g., a human heart failure patient. The term “mammal” as used herein includes but is not limited to a human, non-human primate, dog, cat, mouse, rat, cow, horse, and pig. Mammals other than humans can be advantageously used as subjects that represent animal models of A subject can be male or female. A subject can be one who has been previously diagnosed or identified as having cancer. A subject can be one who has already undergone, or is undergoing, a therapeutic intervention for cancer. A subject can also be one who has not been previously diagnosed as having a cancer; e.g., a subject can be one who exhibits one or more symptoms or risk factors for a cancer, or a subject who does not exhibit symptoms or risk factors for a cancer, or a subject who is asymptomatic for a cancer.

A “sample” in the context of the present teachings refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including gingival crevicular fluid, bone marrow, cerebrospinal fluid (CSF), saliva, mucous, sputum, semen, sweat, urine, or any other bodily fluids. “Blood sample” can refer to whole blood or any fraction thereof, including blood cells, red blood cells, white blood cells or leucocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art. In one embodiment the sample is a whole blood sample.

“Marker,” “markers,” biomarker,” or, “biomarkers,” all refer to a sequence characteristic of a particular variant allele (i.e., polymorphic site) or wild-type allele. A marker can include any allele, including wild-types alleles, SNPs, microsatellites, insertions, deletions, duplications, and translocations. A marker can also include a peptide encoded by an allele comprising nucleic acids. A marker in the context of the present teachings encompasses, without limitation, cytokines, chemokines, growth factors, proteins, peptides, nucleic acids, oligonucleotides, and metabolites, together with their related metabolites, mutations, variants, polymorphisms, modifications, fragments, subunits, degradation products, elements, and other analytes or sample-derived measures. Markers can also include mutated proteins, mutated nucleic acids, variations in copy numbers and/or transcript variants. Markers also encompass non-blood borne factors and non-analyte physiological markers of health status, and/or other factors or markers not measured from samples (e.g., biological samples such as bodily fluids), such as clinical parameters and traditional factors for clinical assessments. Markers can also include any indices that are calculated and/or created mathematically. Markers can also include combinations of any one or more of the foregoing measurements, including temporal trends and differences.

To “analyze” includes measurement and/or detection of data associated with a marker (such as, e.g., presence or absence of a SNP, allele, melting temperature (Tm) or constituent expression levels) in the sample (or, e.g., by obtaining a dataset reporting such measurements, as described below). In some aspects, an analysis can include comparing the measurement and/or detection against a measurement and/or detection in a sample or set of samples from the same subject or other control subject(s). The markers of the present teachings can be analyzed by any of various conventional methods known in the art.

A “dataset” is a set of data (e.g., numerical values) resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring, sequencing, PCR, RT-PCR, microarray, contacting with one or more primers, contacting with one or more probes, antibody binding, or ELISA. The phrase also encompasses receiving a set of data, e.g., from a third party that has processed the sample to experimentally determine the dataset. Additionally, the phrase encompasses mining data from at least one database or at least one publication or a combination of databases and publications.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject's clinical parameters based on a control.

A “nucleotide-based assay” is a nucleic acid-based assay capable of detecting a given nucleic acid sequence of interest, such as in situ hybridization, sequencing, RT-PCR, and hybridization assays that use nucleic acid sequencing. Other examples of nucleotide-based assays include single base extensions (see, e.g., Kobayashi et al, Mol. Cell. Probes, 9:175-182, 1995); single-strand conformation polymorphism analysis, as described, e.g., in Orita et al., Proc. Nat. Acad. Sci. 86, 2766-2770 (1989), allele specific oligonucleotide hybridization (ASO) (e.g., Stoneking et al., Am. J. Hum. Genet. 48:70-382, 1991; Saiki et al., Nature 324, 163-166, 1986; EP 235,726; and WO 89/11548); and sequence-specific amplification or primer extension methods as described in, for example, WO 93/22456; U.S. Pat. Nos. 5,137,806; 5,595,890; 5,639,611; and U.S. Pat. No. 4,851,331; 5′-nuclease assays, as described in U.S. Pat. Nos. 5,210,015; 5,487,972; and 5,804,375; and Holland et al. 1988, Proc. Natl. Acad. Sci. USA 88:7276-7280. Other examples are described in U.S. Pat. Pub. 20110045469, herein incorporated by reference.

### Activin Inhibitors

As used herein, the term activin type II B receptors (ActRIIB) can refer to human activin receptors having accession number NP_001097 or variants thereof, such as those having the arginine at position 64 substituted with alanine. The term soluble ActRIIB (wild type) can refer to the extracellular domain of ActRIIB, e g , amino acids 1 to 134 (with signal sequence), or amino acids 19, 20, 21, 22, 23, 24, or 25 through 134 of SEQ ID NO: 2 (without signal sequence).

Examples of activin inhibitors include those disclosed herein (e.g., variant ActRIIBs such as svActRIIB-based polypeptides), those disclosed in PCT/US2014/14490, those disclosed in PCT/US2012/070571, those disclosed in U.S. Ser. No. 14/085,056, those disclosed in U.S. Pat. No. 7,947,646, those disclosed in U.S. Pat. Nos. 8,486,403, 8,361,957, 8,703,927, 8,343,933, and 8,252,900, and those disclosed in U.S. Ser. Nos. 13/796,135, 13/404,593, 13/403,657, 13/750,249, 13/730,418, and 13/588,468; each of which is herein incorporated by reference, in its entirety, for all purposes.

Further discussion of certain activin inhibitors, such as stabilized receptor polypeptide-based activin inhibitors, follows below.

Stabilized Receptor Polypeptides

The present invention provides an isolated protein comprising a stabilized ActIIB receptor polypeptide (referred herein as “svActRIIB polypeptide”). As used herein the term “svActRIIB protein” refers to a protein comprising a stabilized ActRIIB polypeptide. As used herein the term “isolated” refers to a protein or polypeptide molecule purified to some degree from endogenous material. These polypeptides and proteins are characterized as having the ability to bind and inhibit the activity of any one of activin A, myostatin, or GDF-11, in addition to having improved manufacturability characteristics.

The stabilized ActRIIB polypeptide is characterized by having an amino acid substitution at both position 28 and 44 with respect to SEQ ID NO: 2. For consistency, the amino acid positions on the stabilized ActRIIB polypeptides and proteins are always referred to with respect to the positions in SEQ ID NO: 2, regardless of whether the polypeptide is mature or truncated. As used herein, the term “mature” refers to a polypeptide or peptide without its signal sequence. As used herein, the term “truncated” refers to polypeptides having N terminal amino acids or C terminal amino acids removed.

In one embodiment, the isolated stabilized activin IIB receptor polypeptide (svActRIIB) has the polypeptide sequence set forth in SEQ ID NO: 2, except for a single amino acid substitution at position 28, and a single amino acid substitution at position 44, wherein the substitution at position 28 is selected from W or Y, and the substitution at position 44 is T. In another embodiment, the polypeptide has the sequence set forth in amino acids 19 through 134 of SEQ ID NO: 2, except for a single amino acid substitution at position 28, and a single amino acid substitution at position 44, wherein the substitution at position 28 is selected from W or Y, and the substitution at position 44 is T. In another embodiment, the polypeptide has the sequence set forth in amino acids 23 through 134 of SEQ ID NO: 2, except for a single amino acid substitution at position 28, and a single amino acid substitution at position 44, wherein the substitution at position 28 is selected from W or Y, and the substitution at position 44 is T. In another embodiment, the polypeptide has the sequence set forth in amino acids 25 through 134 of SEQ ID NO: 2, except for a single amino acid substitution at position 28, and a single amino acid substitution at position 44, wherein the substitution at position 28 is selected from W or Y, and the substitution at position 44 is T. In another embodiment, the polypeptide has an amino acid sequence with at least 80%, 85%, 90%, 95%, 96%, 97%, 98% or 99% identity to any one of the polypeptides above, wherein the polypeptide has single amino acid substitution at position 28, and a single amino acid substitution at position 44, wherein the substitution at position 28 is selected from W or Y, and the substitution at position 44 is T, and wherein the polypeptide is capable of binding myostatin, activin A, or GDF-11. In one embodiment, the substitution of the above polypeptides at position 28 is W, and the substitution at position 44 is T, wherein the polypeptide is capable of binding myostatin, activin A, or GDF-11.

In one embodiment, the svActRIIB polypeptide includes a signal sequence, for example, SEQ ID NO: 4, 8, 12, and 16. However, various signal peptides can be used in the preparation of the polypeptides of the instant application. The signal peptides can have the sequence set forth in amino acids 1 to 19 of SEQ ID NO: 4, for example, or the signal sequences set forth in SEQ ID NO: 31 and 32. Any other signal peptides useful for expressing svActRIIB polypeptides may be used. In other embodiments, the signal sequence is removed, leaving the mature peptide. Examples of svActRIIB polypeptides lacking a signal sequence includes, for example, SEQ ID NO: 6, 10, 14 and 18.

In one embodiment, the protein comprises a stabilized activin IIB receptor polypeptide, wherein the polypeptide is selected from the group consisting of polypeptides having the sequence set forth in the group consisting of SEQ ID NO: 4, 6, 12 and 14. These polypeptides represent amino acids 25 to 134 of SEQ ID NO: 2, wherein the polypeptide has single amino acid substitution at position 28, and a single amino acid substitution at position 44, wherein the substitution at position 28 is selected from W or Y, and the substitution at position 44 is T, and wherein the polypeptide is capable of binding myostatin, activin A, or GDF-11, with and without a signal sequence different from that shown in SEQ ID NO: 2. In another embodiment the protein comprises a polypeptide having at least 80%, 85%, 90%, 95%, 96%, 97%, 98%, or 99% sequence identity to SEQ ID NO: 4, 6, 12 or 14, wherein the polypeptide has a W or Y at position 28 and a T at position 44, and wherein the polypeptide is capable of binding myostatin, activin A, or GDF-11. In one embodiment, the substitution at position 28 is W and the substitution at position 44 is T, wherein the polypeptide is capable of binding myostatin, activin A or GDF-11.

In a further embodiment the svActRIIB protein further comprises a heterologous protein. In one embodiment, the heterologous protein is an Fc domain. In a further embodiment, the Fc domain is a human IgG Fc domain. In one embodiment, the protein comprises a polypeptide having the sequence set forth in the group consisting of SEQ ID NO: 8, 10, 16 and 18. In another embodiment, the protein comprises a polypeptide having at least 80%, 85%, 90%, 95%, 96%, 97%, 98%, or 99% sequence identity to SEQ ID NO: 8, 10, 16 or 18, wherein the polypeptide has a W or Y at position 28 and a T at position 44, and wherein the polypeptide is capable of binding myostatin, activin A, or GDF-11. In one embodiment, the substitution at position 28 is W and the substitution at position 44 is T, wherein the polypeptide is capable of binding myostatin, activin A or GDF-11.

In a further embodiment, the protein comprises the any one of the polypeptides described above, wherein the amino acid residue at position 64 is alanine.

In another embodiment, the term svActRIIB polypeptide and protein encompasses proteins comprising fragments of SEQ ID NO: 2, 4, 6, 12 and 14, including N and C terminal truncations, wherein position 28 is W or Y, and position 44 is T, and wherein the polypeptide is capable of binding myostatin, activin A or GDF-11.

As used herein the term “derivative” of the svActRIIB polypeptide refers to the attachment of at least one additional chemical moiety, or at least one additional polypeptide to form covalent or aggregate conjugates such as glycosyl groups, lipids, acetyl groups, or C-terminal or N-terminal fusion polypeptides, conjugation to PEG molecules, and other modifications which are described more fully below. Stabilized ActRIIB receptor polypeptides can also include additional modifications and derivatives, including modifications to the C and N termini which arise from processing due to expression in various cell types such as mammalian cells, E. coli, yeasts and other recombinant host cells.

The svActRIIB proteins of the present invention may further comprise heterologous polypeptides attached to the svActRIIB polypeptide either directly or through a linker sequence to form a fusion protein. As used herein the term “fusion protein” refers to a protein having a heterologous polypeptide attached via recombinant DNA techniques. Heterologous polypeptides include but are not limited to Fc polypeptides, his tags, and leucine zipper domains to promote oligomerization and further stabilization of the stabilized ActRIIB polypeptides as described in, for example, WO 00/29581, which is herein incorporated by reference. In one embodiment, the heterologous polypeptide is an Fc polypeptide or domain. In one embodiment, the Fc domain is selected from a human IgG 1 Fc (SEQ ID NO: 23), modified IgG1 Fc (SEQ ID NO: 47), IgG2 Fc (SEQ ID NO: 22), and IgG4 Fc (SEQ ID NO: 24) domain. The svActRIIB protein can further comprise all or a portion of the hinge sequence of the IgG1 (SEQ ID NO: 29), IgG2 (SEQ ID NO: 28), or IgG4 (SEQ ID NO: 30). Exemplary svActRIIB polypeptides are selected from polypeptides consisting of the sequences as set forth in SEQ ID NO: 8, 10, 16 and 18, as well as those polypeptides having substantial similarity to these sequences, wherein the substitutions at positions 28 and 44 are retained. As used herein, “substantial similarity” refers to sequences that are at least 80% identical, 85% identical, 90% identical, 95% identical, 96% identical, 97% identical, 98% identical, 99% identical to any of SEQ ID NO: 8, 10, 16, and 18, wherein the polypeptides retain W or Y at position 28 and T at position 44, and wherein the polypeptide is capable of binding myostatin, activin A or GDF-11. In one embodiment, the substitution at position 28 is W and the substitution at position 44 is T, wherein the polypeptide is capable of binding myostatin, activin A or GDF-11.

The svActRIIB polypeptide can optionally further comprise a “linker” sequence. Linkers serve primarily as a spacer between a polypeptide and a second heterologous polypeptide or other type of fusion or between two or more stabilized ActRIIB polypeptides. In one embodiment, the linker is made up of amino acids linked together by peptide bonds, preferably from 1 to 20 amino acids linked by peptide bonds, wherein the amino acids are selected from the 20 naturally occurring amino acids. One or more of these amino acids may be glycosylated, as is understood by those of skill in the art. In one embodiment, the 1 to 20 amino acids may be selected from glycine, alanine, proline, asparagine, glutamine, and lysine. In one embodiment, a linker is made up of a majority of amino acids that are sterically unhindered, such as glycine and alanine. Exemplary linkers are polyglycines (particularly (Gly)₅, (Gly)₈, poly(Gly-Ala), and polyalanines. One exemplary suitable linker as shown in the Examples below is (Gly)₄Ser (SEQ ID NO: 25). In a further embodiment, svActRIIB can comprise a “hinge linker”, that is a linker sequence provided adjacent to a hinge region or a partial hinge region of an IgG, as exemplified in SEQ ID NO: 27. Hinge sequences include IgG2Fc (SEQ ID NO: 28), IgG1Fc (SEQ ID NO: 29), and IgG4Fc (SEQ

ID NO: 30).

Hinge linker sequences may also be designed to improve manufacturability and stability of the svActRIIB-Fc proteins. In one embodiment, the hinge linkers of SEQ ID NO: 27, 38, 40, 42, 44, 45, and 46 are designed to improve manufacturability with the IgG2 Fc (SEQ ID NO: 22) when attached to svActRIIB polypeptides. In one embodiment, the hinge linker sequences is designed to improve manufacturability when attaching svActRIIB polypeptides to a human IgG1 Fc (SEQ ID NO: 23) or a modified human IgG1 Fc (SEQ ID NO: 47), for example, the hinge linkers having SEQ ID NO: 48, SEQ ID NO: 49 and SEQ ID NO: 50. The improved manufacturability of these polypeptides is described below in Example 4.

Linkers may also be non-peptide linkers. For example, alkyl linkers such as —NH—(CH₂)s-C(O)—, wherein s=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc.

The svActRIIB polypeptides disclosed herein can also be attached to a non-polypeptide molecule for the purpose of conferring desired properties such as reducing degradation and/or increasing half-life, reducing toxicity, reducing immunogenicity, and/or increasing the biological activity of the svActRIIB polypeptides. Exemplary molecules include but are not limited to linear polymers such as polyethylene glycol (PEG), polylysine, a dextran; a lipid; a cholesterol group (such as a steroid); a carbohydrate, or an oligosaccharide molecule.

The svActRIIB proteins and polypeptides have improved manufacturability properties when compared to other ActRIIB soluble polypeptides. As used herein, the term “manufacturability” refers to the stability of a particular protein during recombinant expression and purification of that protein. Manufacturability is believed to be due to the intrinsic properties of the molecule under conditions of expression and purification. Examples of improved manufacturability characteristics are set forth in the Examples below and include uniform glycosylation of a protein (Example 2), increased cell titer, growth and protein expression during recombinant production of the protein (Example 1), improved purification properties (Example 2), and improved stability at low pH (Example 2). The svActRIIB proteins and polypeptides of the present invention demonstrate the improved manufacturability, along with retention of in vitro and in vivo activity (Examples 2 and 3), compared with other soluble ActRIIB polypeptides. Further, additional hinge linker sequences may confer additional manufacturability benefits, as shown in Example 4 below.

As used herein, the term a “svActRIIB polypeptide activity” or “a biological activity of a soluble ActRIIB polypeptide” refers to one or more in vitro or in vivo activities of the svActRIIB polypeptides including but not limited to those demonstrated in the Example below. Activities of the svActRIIB polypeptides include, but are not limited to, the ability to bind to myostatin or activin A or GDF-11, and the ability to inhibit or neutralize an activity of myostatin or activin A or GDF-11. As used herein, the term “capable of binding” to myostatin, activin A, or GDF-11 refers to binding measured by methods known in the art, such as the KinExA™ method shown in the Examples below. In vitro inhibition of myostatin, activin A, or GDF-11 can be measured using, for example, the pMARE C2C12 cell-based assay described in the Examples below. In vivo activity, demonstrated in Example 3 below, is demonstrated by increased lean muscle mass in mouse models. In vivo activities of the svActRIIB polypeptides and proteins include but are not limited to increasing body weight, increasing lean muscle mass, and increasing the ratio of lean muscle to fat mass. Therapeutic activities further include reducing or preventing cachexia caused by certain types of tumors, preventing the growth of certain types of tumors, and increasing survival of certain animal models. Further discussion of the svActRIIB protein and polypeptide activities is provided below.

In another aspect, the present invention provides an isolated nucleic acid molecule comprising a polynucleotide encoding an svActRIIB polypeptide of the present invention. As used herein the term “isolated” refers to nucleic acid molecules purified to some degree from endogenous material.

In one embodiment, the polynucleotide encodes a polypeptide having the sequence set forth in SEQ ID NO: 2, except for a single amino acid substitution at position 28, and a single amino acid substitution at position 44, wherein the substitution at position 28 is selected from W or Y, and the substitution at position 44 is T. In another embodiment, the polynucleotide encodes a polypeptide having the sequence set forth in amino acids 19 through 134 of SEQ ID NO: 2, except for a single amino acid substitution at position 28, and a single amino acid substitution at position 44, wherein the substitution at position 28 is selected from W or Y, and the substitution at position 44 is T. In another embodiment, the polynucleotide encodes a polypeptide having the sequence set forth in amino acids 23 through 134 of SEQ ID NO: 2, except for a single amino acid substitution at position 28, and a single amino acid substitution at position 44, wherein the substitution at position 28 is selected from W or Y, and the substitution at position 44 is T. In another embodiment, the polynucleotide encodes a polypeptide having the sequence set forth in amino acids 25 through 134 of SEQ ID NO: 2, except for a single amino acid substitution at position 28, and a single amino acid substitution at position 44, wherein the substitution at position 28 is selected from W or Y, and the substitution at position 44 is T. In another embodiment, the polynucleotide encodes the a polypeptide having an amino acid sequence at least 80%, 85%, 90%, 95%, 98% or 99% identity to any one of the polypeptides above, wherein the polypeptide has single amino acid substitution at position 28, and a single amino acid substitution at position 44, wherein the substitution at position 28 is selected from W or Y, and the substitution at position 44 is T, and wherein the polypeptide is capable of binding myostatin, activin A, or GDF-11. In one embodiment, the polynucleotide of the above embodiments encodes a polypeptide wherein the substitution at position 28 is W and the substitution at position 44 is T.

In one embodiment, the isolated nucleic acid molecule of the present invention comprises a polynucleotide encoding a polypeptide having the sequence set forth in the group consisting of SEQ ID NO: 4, 6, 12, and 14. In another embodiment, the nucleic acid comprises a polynucleotide encoding a polypeptide having at least 80%, 90%, 95%, 96%, 97%, 98%, 99% sequence identity to SEQ ID NO: 4, 6, 12 or 14, wherein the polypeptide has a W or Y at position 28 and a T at position 44, and wherein the polypeptide is capable of binding activin A, GDF-11, or myostatin. In one embodiment, the polynucleotide of the above embodiments encodes a polypeptide wherein the substitution at position 28 is W and the substitution at position 44 is T, and wherein the polypeptide is capable of binding activin A, GDF-11 or myostatin.

In another embodiment, the isolated nucleic acid molecule further comprises a polynucleotide encoding at least one heterologous protein. In one embodiment, the heterologous protein is an Fc domain, in a further embodiment, the Fc domain is a human IgG Fc domain. In another embodiment, the nucleic acid molecule further comprises polynucleotides encoding the linkers and hinge linkers set forth in SEQ ID NO: 25, 27, 38, 40, 42, 44, 45, 46, 48, 49 or 50. In a further embodiment, such polynucleotides have sequences selected from the group consisting of SEQ ID NO: 26, 37, 39, 41, and 43.

In one embodiment, the nucleic acid molecule comprises a polynucleotide encoding a polypeptide consisting of the sequence set forth in the group consisting of SEQ ID NO: 8, 10, 16 and 18. In another embodiment, the nucleic acid comprises a polynucleotide encoding a polypeptide having at least 80%, 90%, 95%, 96%, 97%, 98%, 99% sequence identity to the group consisting of SEQ ID NO: 8, 10, 16 and 18, wherein the polypeptide has a W or Y at position 28 and a T at position 44, and wherein the polypeptide is capable of binding activin A, GDF-11, or myostatin. In one embodiment, the polynucleotide of the above embodiments encodes a polypeptide wherein the substitution at position 28 is W and the substitution at position 44 is T, and wherein the polypeptide is capable of binding myostatin, activin A or GDF-11.

In one embodiment, the isolated nucleic acid molecule comprises a polynucleotide having the sequence selected from the group consisting of SEQ ID NO: 3, 5, 11 or 13, or its complement. In another embodiment, the isolated nucleic acid molecule comprises a polynucleotide having the sequence selected from the group consisting of the sequence SEQ ID NO: 7, 9, 15 and 17, or its complement. In a further embodiment the isolated nucleic acid molecule hybridizes under stringent or moderate conditions with SEQ ID NO: 3, 5, 7, 9, 11, 13, 15 or 17 wherein the encoded polypeptide is substantially similar to SEQ ID NO: 4, 6, 8, 10, 12, 14, 16, or 18, wherein the polypeptide comprises an amino acid sequence having W or Y at position 28, and T at position 44, and wherein the encoded polypeptide is capable of binding or inhibiting activin A, myostatin or GDF-11.

Nucleic acid molecules of the invention include DNA in both single-stranded and double-stranded form, as well as the RNA complement thereof DNA includes, for example, cDNA, genomic DNA, synthetic DNA, DNA amplified by PCR, and combinations thereof. Genomic DNA may be isolated by conventional techniques, such as by using the DNA of SEQ ID NO: 3, 5, 11 or 13, or a suitable fragment thereof, as a probe. Genomic DNA encoding activin inhibitor polypeptides is obtained from genomic libraries which are available for a number of species. Synthetic DNA is available from chemical synthesis of overlapping oligonucleotide fragments followed by assembly of the fragments to reconstitute part or all of the coding regions and flanking sequences. RNA may be obtained from procaryotic expression vectors which direct high-level synthesis of mRNA, such as vectors using T7 promoters and RNA polymerase. cDNA is obtained from libraries prepared from mRNA isolated from various tissues that express ActRIIB. The DNA molecules of the invention include full length genes as well as polynucleotides and fragments thereof. The full length gene may also include sequences encoding the N-terminal signal sequence.

The invention further provides the nucleic acid molecule describe above, wherein the polynucleotide is operably linked to a transcriptional or translational regulatory sequence.

In another aspect of the present invention, expression vectors containing the nucleic acid molecules and polynucleotides of the present invention are also provided, and host cells transformed with such vectors, and methods of producing the activin inhibitor polypeptides are also provided. The term “expression vector” refers to a plasmid, phage, virus or vector for expressing a polypeptide from a polynucleotide sequence. Vectors for the expression of the activin inhibitor polypeptides contain at a minimum sequences required for vector propagation and for expression of the cloned insert. An expression vector comprises a transcriptional unit comprising an assembly of (1) a genetic element or elements having a regulatory role in gene expression, for example, promoters or enhancers, (2) a sequence that encodes activin inhibitor polypeptides and proteins to be transcribed into mRNA and translated into protein, and (3) appropriate transcription initiation and termination sequences. These sequences may further include a selection marker. Vectors suitable for expression in host cells are readily available and the nucleic acid molecules are inserted into the vectors using standard recombinant DNA techniques. Such vectors can include promoters which function in specific tissues, and viral vectors for the expression of activin inhibitor polypeptides in targeted human or animal cells. An exemplary expression vector suitable for expression of an activin inhibitor is the pDSRa, (described in WO 90/14363, herein incorporated by reference) and its derivatives, containing activin inhibitor polynucleotides, as well as any additional suitable vectors known in the art or described below.

The invention further provides methods of making activin inhibitor polypeptides. A variety of other expression/host systems may be utilized. These systems include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells useful in recombinant protein production include but are not limited to VERO cells, HeLa cells, Chinese hamster ovary (CHO) cell lines, or their derivatives such as Veggie CHO and related cell lines which grow in serum-free media (see Rasmussen et al., 1998, Cytotechnology 28:31) or CHO strain DX-B11, which is deficient in DHFR (see Urlaub et al., 1980, Proc. Natl. Acad. Sci. USA 77:4216-20) COS cells such as the COS-7 line of monkey kidney cells (ATCC CRL 1651) (see Gluzman et al., 1981, Cell 23:175), W138, BHK, HepG2, 3T3 (ATCC CCL 163), RIN, MDCK, A549, PC12, K562, L cells, C127 cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Mammalian expression allows for the production of secreted or soluble polypeptides which may be recovered from the growth medium.

Using an appropriate host-vector system, activin inhibitor polypeptides are produced recombinantly by culturing a host cell transformed with an expression vector containing the nucleic acid molecules of the present invention under conditions allowing for production. Transformed cells can be used for long-term, high-yield polypeptide production. Once such cells are transformed with vectors that contain selectable markers as well as the desired expression cassette, the cells can be allowed to grow in an enriched media before they are switched to selective media, for example. The selectable marker is designed to allow growth and recovery of cells that successfully express the introduced sequences. Resistant clumps of stably transformed cells can be proliferated using tissue culture techniques appropriate to the cell line employed. An overview of expression of recombinant proteins is found in Methods of Enzymology, v. 185, Goeddell, D. V., ed., Academic Press (1990).

In some cases, such as in expression using procaryotic systems, the expressed polypeptides of this invention may need to be “refolded” and oxidized into a proper tertiary structure and disulfide linkages generated in order to be biologically active. Refolding can be accomplished using a number of procedures well known in the art. Such methods include, for example, exposing the solubilized polypeptide to a pH usually above 7 in the presence of a chaotropic agent. The selection of chaotrope is similar to the choices used for inclusion body solubilization, however a chaotrope is typically used at a lower concentration. Exemplary chaotropic agents are guanidine and urea. In most cases, the refolding/oxidation solution will also contain a reducing agent plus its oxidized form in a specific ratio to generate a particular redox potential which allows for disulfide shuffling to occur for the formation of cysteine bridges. Some commonly used redox couples include cysteine/cystamine, glutathione/dithiobisGSH, cupric chloride, dithiothreitol DTT/dithiane DTT, and 2-mercaptoethanol (bME)/dithio-bME. In many instances, a co-solvent may be used to increase the efficiency of the refolding. Commonly used cosolvents include glycerol, polyethylene glycol of various molecular weights, and arginine.

In addition, the polypeptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d.Ed., Pierce Chemical Co. (1984); Tam et al., J Am Chem Soc, 105:6442, (1983); Merrifield, Science 232:341-347 (1986); Barany and Merrifield, The Peptides, Gross and Meienhofer, eds, Academic Press, New York, 1-284; Barany et al., Int J Pep Protein Res, 30:705-739 (1987).

The polypeptides and proteins of the present invention can be purified according to protein purification techniques are well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The term “isolated polypeptide” or “purified polypeptide” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography, isoelectric focusing, gel electrophoresis, and combinations of these techniques. As is generally known in the art, it is believed that the order of conducting the various purification steps may be changed, or that certain steps may be omitted, and still result in a suitable method for the preparation of a substantially purified polypeptide. Exemplary purification steps are provided in the Examples below.

Various methods for quantifying the degree of purification of polypeptide will be known to those of skill in the art in light of the present disclosure. These include, for example, determining the specific binding activity of an active fraction, or assessing the amount of peptide or polypeptide within a fraction by SDS/PAGE analysis. A preferred method for assessing the purity of a polypeptide fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the polypeptide or peptide exhibits a detectable binding activity.

Stabilized activin type IIB polypeptides bind to ligands that activate muscle-degradation cascades. svActRIIB polypeptides capable of binding and inhibiting the activity of the ligands activin A, myostatin, and/or GDF-11, and have the ability to treat diseases that involve muscle atrophy, as well as the treatment of certain cancers, and other diseases. The Examples below show improved properties for svActRIIB polypeptides and proteins having the amino acid substitutions described herein, while retaining the ability to bind and neutralize myostatin, activin A, or GDF-11 in in vitro assays, as well as retaining in vivo activity. These properties result in proteins and polypeptides having improved manufacturability in comparison to other soluble receptors.

### Pharmaceutical Compositions

Pharmaceutical compositions comprising one or more activin inhibitors are also provided. Such compositions comprise a therapeutically or prophylactically effective amount of the polypeptide or protein in admixture with pharmaceutically acceptable materials, and physiologically acceptable formulation materials. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company, 1990).

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the polypeptide. For example, suitable compositions may be water for injection, physiological saline solution for parenteral administration.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffers, or acetate buffers, which may further include sorbitol or a suitable substitute thereof. In one embodiment of the present invention, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose.

The formulations can be delivered in a variety of methods, for example, by inhalation therapy, orally, or by injection. When parenteral administration is contemplated, the therapeutic compositions for use in this invention may be in the form of a pyrogen-free, parenterally acceptable aqueous solution comprising the desired polypeptide in a pharmaceutically acceptable vehicle. A particularly suitable vehicle for parenteral injection is sterile distilled water in which a polypeptide is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In another aspect, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions. In another embodiment, a pharmaceutical composition may be formulated for inhalation. Inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

It is also contemplated that certain formulations may be administered orally. In one embodiment of the present invention, molecules that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the therapeutic molecule. Diluents, flavorings, low melting point waxes, vegetable oils, lubricants, suspending agents, tablet disintegrating agents, and binders may also be employed. Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Additional pharmaceutical compositions will be evident to those skilled in the art, including formulations involving polypeptides in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 that describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556 (1983), poly (2-hydroxyethyl-methacrylate) (Langer et al., J. Biomed. Mater. Res., 15:167-277, (1981); Langer et al., Chem. Tech.,12:98-105(1982)), ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., PNAS (USA), 82:3688 (1985); EP 36,676; EP 88,046; EP 143,949.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the polypeptide is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. Polypeptide compositions may be preferably injected or administered intravenously. Long-acting pharmaceutical compositions may be administered every three to four days, every week, or biweekly depending on the half-life and clearance rate of the particular formulation. The frequency of dosing will depend upon the pharmacokinetic parameters of the polypeptide in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, or intraperitoneal; as well as intranasal, enteral, topical, sublingual, urethral, vaginal, or rectal means, by sustained release systems or by implantation devices. Where desired, the compositions may be administered by bolus injection or continuously by infusion, or by implantation device. Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

In some cases, the vActRIIB polypeptides of the present invention can be delivered by implanting certain cells that have been genetically engineered, using methods such as those described herein, to express and secrete the polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the polypeptide product(s) but prevent the destruction of the cells by the patient's immune system or by other detrimental factors from the surrounding tissues.

vActRIIB gene therapy in vivo is also envisioned wherein a nucleic acid molecule encoding vActRIIB, or a derivative of vActRIIB is introduced directly into the subject. For example, a nucleic acid sequence encoding a vActRIIB is introduced into target cells via local injection of a nucleic acid construct with or without an appropriate delivery vector, such as an adeno-associated virus vector. Alternative viral vectors include, but are not limited to, retroviruses, adenovirus, herpes simplex virus, and papilloma virus vectors. Physical transfer of the virus vector may be achieved in vivo by local injection of the desired nucleic acid construct or other appropriate delivery vector containing the desired nucleic acid sequence, liposome-mediated transfer, direct injection (naked DNA), or microparticle bombardment (gene-gun).

The compositions of the present disclosure may be used alone or in combination with other therapeutic agents to enhance their therapeutic effects or decrease potential side effects.

Uses of vActRIIB Compositions

The present invention provides methods and pharmaceutical compositions for reducing or neutralizing the amount or activity of myostatin, activin A, or GDF-11 in vivo and in vitro. Activin inhibitors have a high binding affinity for myostatin, activin A, and GDF-11, and are capable of reducing and inhibiting the biological activities of at least one of myostatin, activin A and GDF-11.

In one aspect, the present invention provides methods and reagents for treating myostatin-related and/or activin A related disorders in a subject in need of such a treatment by administering an effective dosage of an activin inhibitor composition to the subject. As used herein the term “subject” refers to any animal, such as mammals including humans.

The compositions of the present invention are useful for increasing lean muscle mass in a subject. The compositions may also be useful to increase lean muscle mass in proportion to fat mass, and thus decrease fat mass as percentage of body weight in a subject. Example 3 demonstrates that the vActRIIB polypeptides and proteins of the invention can increase lean muscle mass in animals.

The disorders that can be treated by an activin inhibitor composition include but are not limited to various forms of muscle wasting, as well as metabolic disorders such as diabetes and related disorders, and bone degenerative diseases such as osteoporosis.

Muscle wasting disorders also include dystrophies such as Duchenne's muscular dystrophy, progressive muscular dystrophy, Becker's type muscular dystrophy, Dejerine-Landouzy muscular dystrophy, Erb's muscular dystrophy, and infantile neuroaxonal muscular dystrophy. Additional muscle wasting disorders arise from chronic diseases or disorders such as amyotrophic lateral sclerosis, congestive obstructive pulmonary disease, cancer, AIDS, renal failure, organ atrophy, androgen deprivation, and rheumatoid arthritis.

Over-expression of myostatin and/or activin may contribute to cachexia, a severe muscle wasting syndrome. Cachexia results from cancers, and also arises due to rheumatoid arthritis, diabetic nephropathy, renal failure, chemotherapy, injury due to burns, as well as other causes. In another example, serum and intramuscular concentrations of myostatin-immunoreactive protein was found to be increased in men exhibiting AIDS-related muscle wasting and was inversely related to fat-free mass (Gonzalez-Cadavid et al., PNAS USA 95: 14938-14943 (1998)). Myostatin levels have also been shown to increase in response to burns injuries, resulting in a catabolic muscle effect (Lang et al, FASEB J 15, 1807-1809 (2001)). Additional conditions resulting in muscle wasting may arise from inactivity due to disability such as confinement in a wheelchair, prolonged bed rest due to stroke, illness, spinal chord injury, bone fracture or trauma, and muscular atrophy in a microgravity environment (space flight). For example, plasma myostatin immunoreactive protein was found to increase after prolonged bed rest (Zachwieja et al. J Gravit Physiol. 6(2):11(1999). It was also found that the muscles of rats exposed to a microgravity environment during a space shuttle flight expressed an increased amount of myostatin compared with the muscles of rats which were not exposed (Lalani et al., J.Endocrin 167 (3):417-28 (2000)).

In addition, age-related increases in fat to muscle ratios, and age-related muscular atrophy appear to be related to myostatin. For example, the average serum myostatin-immunoreactive protein increased with age in groups of young (19-35 yr. old), middle-aged (36-75 yr. old), and elderly (76-92 yr old) men and women, while the average muscle mass and fat-free mass declined with age in these groups (Yarasheski et al. J Nutr Aging 6(5):343-8 (2002)). In addition, myostatin has now been found to be expressed at low levels in heart muscle and expression is upregulated in cardiomyocytes after infarct (Sharma et al., J Cell Physiol. 180 (1):1-9 (1999)). Therefore, reducing myostatin levels in the heart muscle may improve recovery of heart muscle after infarct.

Myostatin also appears to influence metabolic disorders including type 2 diabetes, noninsulin-dependent diabetes mellitus, hyperglycemia, and obesity. For example, lack of myostatin has been shown to improve the obese and diabetic phenotypes of two mouse models (Yen et al. FASEB J. 8:479 (1994). The activin inhibitors of the present disclosure are suitable for treating such metabolic disorders. Therefore, administering the compositions of the present invention will improve diabetes, obesity, and hyperglycemic conditions in suitable subjects. In addition, compositions containing activin inhibitors may decrease food intake in obese individuals.

Administering activin inhibitors may improve bone strength and reduce osteoporosis and other degenerative bone diseases. It has been found, for example, that myostatin-deficient mice showed increased mineral content and density of the mouse humerus and increased mineral content of both trabecular and cortical bone at the regions where the muscles attach, as well as increased muscle mass (Hamrick et al. Calcif Tissue Int 71(1):63-8 (2002)). In addition, activin inhibitor compositions of the present invention can be used to treat the effects of androgen deprivation in cases such as androgen deprivation therapy used for the treatment of prostate cancer, for example.

The present invention also provides methods and compositions for increasing muscle mass in food animals by administering an effective dosage of the activin inhibitors to the animal. Since the mature C-terminal myostatin polypeptide is similar or identical in all species tested, activin inhibitors would be expected to be effective for increasing lean muscle mass and reducing fat in any agriculturally important species including cattle, chicken, turkeys, and pigs.

The activin inhibitor polypeptides and compositions of the present invention also antagonize the activity of activin A, as shown in the in vitro assays below. Activin A is known to be expressed in certain types of cancers, particularly gonadal tumors such as ovarian carcinomas, and to cause severe cachexia. (Ciprano et al. Endocrinol 141 (7):2319-27 (2000), Shou et al., Endocrinol 138 (11):5000-5 (1997); Coerver et al, Mol Endocrinol 10(5):534-43 (1996); Ito et al. British J Cancer 82(8):1415-20 (2000), Lambert-Messerlian, et al, Gynecologic Oncology 74:93-7 (1999). Therefore, the compositions of the present disclosure may be used to treat conditions related to activin A overexpression, as well as myostatin expression, such as cachexia from certain cancers and the treatment of certain gonadal type tumors.

In addition, the activin inhibitor polypeptides of the present invention are useful for detecting and quantitating myostatin, activin A, or GDF-11 in any number of assays. In general, the activin inhibitors of the present invention are useful as capture agents to bind and immobilize myostatin, activin A, or GDF-11 in a variety of assays, similar to those described, for example, in Asai, ed., Methods in Cell Biology, 37, Antibodies in Cell Biology, Academic Press, Inc., New York (1993). The polypeptides may be labeled in some manner or may react with a third molecule such as an antibody which is labeled to enable myostatin to be detected and quantitated. For example, a polypeptide or a third molecule can be modified with a detectable moiety, such as biotin, which can then be bound by a fourth molecule, such as enzyme-labeled streptavidin, or other proteins. (Akerstrom, J Immunol 135:2589 (1985); Chaubert, Mod Pathol 10:585 (1997)).

### Detection Assays

Nucleic Acids

Nucleic acid molecules can be RNA, for example, mRNA, or DNA, such as cDNA and genomic DNA. DNA molecules can be double-stranded or single-stranded; single-stranded RNA or DNA can be the coding, or sense, strand or the non-coding, or antisense strand. The nucleic acid molecule can include all or a portion of the coding sequence of the gene and can further comprise additional non-coding sequences such as introns and non-coding 3′ and 5′ sequences (including regulatory sequences, for example).

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid molecule comprises at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb but not limited to 4 kb, 3 kb, 2 kb, 1 kb, 0.5 kb or 0.1 kb of nucleotides which flank the nucleic acid molecule in the genomic DNA of the cell from which the nucleic acid molecule is derived.

An isolated nucleic acid molecule can include a nucleic acid molecule or nucleic acid sequence that is synthesized chemically or by recombinant means. Such isolated nucleic acid molecules are useful as probes for isolating homologous sequences (e.g., from other mammalian species), for gene mapping (e.g., by in situ hybridization with chromosomes), or for detecting expression of the gene in tissue (e.g., human tissue), such as by Northern or Southern blot analysis.

Nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one aspect, the invention includes variants described herein that hybridize under high stringency hybridization conditions (e.g., for selective hybridization) to a nucleotide sequence encoding an amino acid sequence or a polymorphic variant thereof.

Such nucleic acid molecules can be detected and/or isolated by specific hybridization (e.g., under high stringency conditions). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 90%, 95%). For example, certain high stringency conditions can be used which distinguish perfectly complementary nucleic acids from those of less complementarity. “High stringency conditions,” “moderate stringency conditions” and “low stringency conditions,” as well as methods for nucleic acid hybridizations are explained on pages 2.10.1-2.10.16 and pages 6.3.1-6.3.6 in Current Protocols in Molecular Biology (Ausubel, F. et al., “Current Protocols in Molecular Biology”, John Wiley & Sons, (1998)), and in Kraus, M. and Aaronson, S., Methods Enzymol., 200:546-556 (1991), incorporated herein, by reference.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain aspects, the length of a sequence aligned for comparison purposes is at least 30%, for example, at least 40%, in certain aspects at least 60%, and in other aspects at least 70%, 80%, 90% or 95% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., W=5 or W=20).

The present invention also provides isolated nucleic acid molecules that contain a fragment or portion that hybridizes under highly stringent conditions to a nucleotide sequence or the complement of such a sequence, and also provides isolated nucleic acid molecules that contain a fragment or portion that hybridizes under highly stringent conditions to a nucleotide sequence encoding an amino acid sequence or polymorphic variant thereof. The nucleic acid fragments of the invention are at least about 15, preferably at least about 18, 20, 23 or 25 nucleotides, and can be 30, 40, 50, 100, 200 or more nucleotides in length.

The nucleic acid molecules of the invention can be identified and isolated using standard molecular biology techniques and the sequence information provided herein. For example, nucleic acid molecules can be amplified and isolated by the polymerase chain reaction (PCR) using synthetic oligonucleotide primers designed based on the sequence of a nucleic acid sequence of interest or the complement of such a sequence, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis et al., Academic Press, San Diego, Calif., 1990); Mattila et al., Nucl. Acids Res. 19: 4967 (1991); Eckert et al., PCR Methods and Applications 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci. USA 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled, for example, radiolabeled, and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Additionally, fluorescence methods are also available for analyzing nucleic acids (Chen et al., Genome Res. 9, 492 (1999)) and polypeptides. Using these or similar methods, the polypeptide and the DNA encoding the polypeptide can be isolated, sequenced and further characterized.

The nucleic acid sequences can also be used to compare with endogenous DNA sequences in patients to identify one or more of the disorders, and as probes, such as to hybridize and discover related DNA sequences or to subtract out known sequences from a sample. The nucleic acid sequences can further be used to derive primers for genetic fingerprinting. Portions or fragments of the nucleotide sequences identified herein (and the corresponding complete gene sequences) can be used in numerous ways, such as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. The nucleic acid sequences can additionally be used as reagents in the screening and/or diagnostic assays described herein, and can also be included as components of kits (e.g., reagent kits) for use in the screening and/or diagnostic assays described herein.

Probes and Primers

In some embodiments and as described in the examples, the methods of the invention employ probes or primers in assays such as those described herein. In some embodiments, the invention includes compositions comprising synthetic probes or primers described herein.

“Probes” or “primers” are oligonucleotides that hybridize in a base-specific manner to a complementary strand of nucleic acid molecules. Such probes and primers include polypeptide nucleic acids, as described in Nielsen et al., Science 254:1497-1500 (1991).

A probe or primer comprises a region of nucleotide sequence that hybridizes to at least about 15, for example about 20-25, and in certain aspects about 40, 50 or 75, consecutive nucleotides of a nucleic acid molecule comprising a contiguous nucleotide sequence of or polymorphic variant thereof. In other aspects, a probe or primer comprises 100 or fewer nucleotides, in certain aspects from 6 to 50 nucleotides, for example from 12 to 30 nucleotides. In other aspects, the probe or primer is at least 70% identical to the contiguous nucleotide sequence or to the complement of the contiguous nucleotide sequence, for example at least 80% identical, in certain aspects at least 90% identical, and in other aspects at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

In some embodiments, a peptide nucleic acid (PNA) probe can be used instead of a nucleic acid probe in the hybridization methods. PNA is a DNA mimic having a peptide-like, inorganic backbone, such as N-(2-aminoethyl) glycine units, with an organic base (A, G, C, T or U) attached to the glycine nitrogen via a methylene carbonyl linker (see, for example, Nielsen, P. E. et al., Bioconjugate Chemistry 5, American Chemical Society, p. 1 (1994). The PNA probe can be designed to specifically hybridize to a nucleic acid.

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′end, or in the middle), the Tm could be increased considerably.

Assays and Kits

Nucleic acids, probes, primers, and antibodies such as those described herein can be used in a variety of methods, as well as in kits. Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies which bind to altered or to non-altered (native) polypeptide, means for amplification of nucleic acids comprising a nucleic acid or for a portion of , or means for analyzing the nucleic acid sequence of a nucleic acid or for analyzing the amino acid sequence of a polypeptide as described herein, etc. .

In one aspect of the invention, hybridization methods are used, such as Southern analysis, Northern analysis, or in situ hybridizations, (see Current Protocols in Molecular Biology, Ausubel, F. et al., eds, John Wiley & Sons, including all supplements through 1999).

In another aspect, arrays of oligonucleotide probes that are complementary to target nucleic acid sequence segments from an individual can be used to identify a nucleic acid. For example, in one aspect, an oligonucleotide array can be used. Oligonucleotide arrays typically comprise a plurality of different oligonucleotide probes that are coupled to a surface of a substrate in different known locations. These oligonucleotide arrays have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186, the entire teachings of which are incorporated by reference herein. Techniques for the synthesis of these arrays using mechanical synthesis methods are described in, e.g., U.S. Pat. No. 5,384,261; the entire teachings are incorporated by reference herein. In another example, linear arrays can be utilized.

Once an oligonucleotide array is prepared, a nucleic acid of interest is hybridized with the array and scanned. Hybridization and scanning are generally carried out by methods described herein and also in, e.g., published PCT Application Nos. WO 92/10092 and WO 95/11995, and U.S. Pat. No. 5,424,186, the entire teachings of which are incorporated by reference herein. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

In one aspect of the invention, genotypic information is obtained by expression analysis by quantitative PCR (kinetic thermal cycling). This technique, utilizing TaqMan assays, can assess the presence of an alteration in the expression or composition of the polypeptide encoded by a nucleic acid or splicing variants encoded by a nucleic acid. TaqMan probes can also be used to allow the identification of nucleic acids. Further, the expression of the variants can be quantified as physically or functionally different.

In another aspect of the invention, genotypic information is obtained is made by examining expression and/or composition of a polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by a nucleic acid, or for the presence of a particular variant encoded by a nucleic acid. An alteration in expression of a polypeptide encoded by a nucleic acid can be, for example, an alteration in the quantitative polypeptide expression (i.e., the amount of polypeptide produced); an alteration in the composition of a polypeptide encoded by a nucleic acid is an alteration in the qualitative polypeptide expression (e.g., expression of an altered polypeptide or of a different splicing variant). In a preferred aspect, diagnosis of a susceptibility to a sudden cardiac event can be made by detecting a particular splicing variant encoded by that nucleic acid, or a particular pattern of splicing variants.

### Uses of Assays for Informing Treatment of a Subject

The methods disclosed herein can be employed together with methods of treatment of a disease, e.g., cancer. Typically a cancer to be treated will be one that tests positive for the markers detected using the various detection methods disclosed herein. Examples of cancers that can be treated include those described in the tables in the Examples section, e.g., ovary, endometrial, pancreas, bile duct, lung, gastric, head/neck, breast, colorectal, melanoma, and testicular. Generally the treatment used to treat the subject will be an anti-activin compound, e.g., an anti-activin compound having the amino acid sequence shown in SEQ ID NO:10.

Also disclosed herein is a method for determining the likelihood that a subject will respond to treatment with an activin inhibitor, comprising performing an assay on a sample from the subject to generate a dataset comprising data representing the expression of at least two markers associated with activin signaling such as INHBA and ACVR2B. In certain aspects the method can further comprise determining, based on the dataset, the likelihood that the subject will respond to treatment with the activin inhibitor, wherein detectable expression of the at least two markers within the sample indicates that the subject is more likely to be responsive to treatment, and/or wherein the lack of detectable expression of at least one of the at least two markers within the sample indicates that the subject is less likely to be responsive to treatment.

In some aspects, an assay can be a nucleotide-based assay, a protein-based assay, or other assay known in the art. In some aspects, a nucleotide-based assay is an in situ hybridization assay performed using a plurality of distinct probes, optionally wherein the plurality of distinct probes hybridize to the nucleotides located at positions 364-1374 of the nucleotide sequence shown in SEQ ID NO:51 or the nucleotides located at positions 627-1503 of the nucleotide sequence shown in SEQ ID NO:52. In some aspects, a protein-based assay is ELISA, western blot, or FACS.

In some aspects, a sample can comprise one or more cells, one or more cancer cells, RNA from one or more cells or cancer cells, one or more fibroblasts, one or more stromal fibroblasts, stroma, and/or cancer-associated reactive stroma. In certain aspects cancer cells can be ovary, endometrial, pancreas, bile duct, lung, gastric, head/neck, breast, colorectal, melanoma, or testicular cancer cells. In certain aspects a sample can be cell-free.

In some aspects, an activin inhibitor comprises a polypeptide such as a fusion polypeptide (e.g., an Fc conjugate) or an antibody. In certain aspects the activin inhibitor can be those disclosed herein (e.g., variant ActRIIBs such as svActRIIB-based polypeptides), those disclosed in PCT/US2014/14490, those disclosed in PCT/US2012/070571, those disclosed in U.S. Ser. No. 14/085,056, those disclosed in U.S. Pat. No. 7,947,646, those disclosed in U.S. Pat. Nos. 8,486,403, 8,361,957, 8,703,927, 8,343,933, and 8,252,900, and/or those disclosed in U.S. Ser. Nos. 13/796,135, 13/404,593, 13/403,657, 13/750,249, 13/730,418, and 13/588,468. In certain aspects the activin inhibitor is a polypeptide that has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

In some aspects, a subject is a mammalian subject such as a human subject. In some aspects, the subject has a disease such as cancer.

In some aspects, the method further comprises administering the activin inhibitor to the subject. In certain aspects, the activin inhibitor is administered when there is detectable expression of the at least two markers within the sample as this can indicate that the subject is more likely to be responsive to treatment. In certain aspects, the activin inhibitor is not administered or withheld if there is a lack of detectable expression of at least one of the at least two markers within the sample as this can indicate that the subject is less likely to be responsive to treatment. In certain aspects the activin inhibitor can be administered with, before, or after a second treatment such a chemotherapeutic.

In some aspects, the dataset further comprises additional data. In some aspects the additional data can represent the location of the at least two markers relative to each other, e.g., co-localization of the markers. Co-localization refers to the presence of each of at least two markers within a single cell (indicating autocrine signaling) or the presence of each of at least two markers across neighboring cells (indicating paracrine signaling), where neighboring cells can be distinct cells that are in physical contact with one another or distinct cells that are in operable contact with one another. In certain aspects neighboring cells can be cells of the same cell type. In certain aspects, neighboring cells can be cells of distinct cell types, e.g., a cancer cell and a stromal fibroblast. In some aspects, co-localization of the markers indicates that the subject is more likely to be responsive to treatment with the activin inhibitor, optionally wherein the co-localization of the markers is in a single cell indicating autocrine signaling, and optionally wherein the co-localization of the markers is in neighboring cells indicating paracrine signaling. In some aspects, the presence of co-localization in addition to the presence of each marker can further indicate that the subject is more likely to be responsive to treatment with the activin inhibitor, e.g., because the presence of co-localization can indicate the presence of activin-based autocrine and/or paracrine signaling. In some aspects, the absence of co-localization can further indicate that the subject is less likely to be responsive to treatment with the activin inhibitor.

Also disclosed herein is a method for assaying the expression of INHBA and ACVR2B in a sample from a subject, comprising: performing an in situ hybridization assay on the sample using a plurality of distinct probes that hybridize to the nucleotides located at positions 364-1374 of the nucleotide sequence shown in SEQ ID NO:51 or the nucleotides located at positions 627-1503 of the nucleotide sequence shown in SEQ ID NO:52; and determining the expression of INHBA and ACVR2B based on the assay, wherein detectable expression of INHBA and ACVR2B within the sample indicates that the subject is more likely to be responsive to treatment with an activin inhibitor, and/or wherein the lack of detectable expression of at least one of INHBA and ACVR2B within the sample indicates that the subject is less likely to be responsive to treatment with the activin inhibitor. The results of the assay can be used to inform activin inhibitor administration decisions, e.g., as described above.

### Computer Implementation

In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

The invention having been described, the following examples are offered by way of illustration, and not limitation.

### EXAMPLES

### Example 1

### Expression and Purification of svActRIIB Polypeptides

The following methods were used for expressing and purifying the stabilized ActRIIB polypeptides.

The cDNA of the human activin type IIB receptor was isolated from a cDNA library of human testis origin (Clontech, Inc.) and cloned as described in U.S. application Ser. No. 11/590,962, U.S. application publication No: 2007/0117130, which is herein incorporated by reference.

The following method was used to produce the svActRIIB-Fc (E28W, S44T) polypeptide (SEQ ID NO: 10), and the ActRIIB-Fc (E28W) (SEQ ID NO: 21). Polynucleotides encoding the svActRIIB, (E28W, S44T) (SEQ ID NO: 5), or polynucleotides encoding ActRIIB (E28W) (SEQ ID NO: 19) were fused to polynucleotides encoding the human IgG2 Fc (SEQ ID NO: 22), via polynucleotides encoding hinge linker sequence (SEQ ID NO: 26) using PCR overlap extension using primers containing the mutation resulting in the amino acid substitutions at position 28 of E to W, and at position 44 of S to T. The full polynucleotide sequence is SEQ ID NO: 9 for svActRIIB-IgG Fc (E28W, S44T), and SEQ ID NO: 20 for ActRIIB-ActRIIB-IgG Fc (E28W). Double stranded DNA fragments were subcloned into vectors pTTS (Biotechnology Research Institute, National Research Council Canada (NRCC), 6100 Avenue Royalmount, Montréal (Québec) Canada H4P 2R2), pDSRa described in WO/9014363) and/or derivatives of pDSRa.

Transient expression of stabilized ActRIIB-Fc polypeptides was carried out as follows.

The svActRIIB-IgG Fc (E28W, S44T) (SEQ ID NO: 10), and ActRIIB-IgG Fc (E28W) (SEQ ID NO: 21) polypeptides were expressed transiently in serum-free suspension adapted 293-6E cells (National Research Council of Canada, Ottawa, Canada) maintained in FreeStyleTM medium (Invitrogen Corporation, Carlsbad, CA) supplemented with 250 μg/ml geneticin (Invitrogen) and 0.1% Pluronic F68 (Invitrogen). Transfections were performed as 1 L cultures. Briefly, the cell inoculum was grown to 1.1×10⁶ cells/ml in a 4 L fernbach shake flask (Corning, Inc.). The shake flask culture was maintained on an Innova 2150 shaker platform (News Brunswick Scientific, Edison, N.J.) at 65 RPM which was placed in a humidified incubator maintained at 37° C. and 5% CO₂. At the time of transfection, the 293-6E cells were diluted to 1.0×10⁶ cells/ml.

The transfection complexes were formed in 100 ml FreeStyle™ 293 Media (Invitrogen). 1 mg plasmid DNA was first added to the medium followed by 3 ml of FuGene HD transfection reagent (Roche Applied Science, Indianapolis, Ind.). The transfection complex was incubated at room temperature for approximately 15 minutes and then added to the cells in the shake flask. Twenty hours post transfection, 20% (w/v) of peptone TN1 (OrganoTechnie S.A., TeknieScience, QC, Canada) was added to reach a final concentration of 0.5% (w/v). The transfection/expression was performed for 4-7 days, after which the conditioned medium was harvested by centrifugation at 4,000 RPM for 60 minutes at 4° C.

Stable transfection and expression was carried out as follows. The svActRIIB-IgG-Fc cell lines were created by transfecting stable CHO host cells with the expression plasmids containing polynucleotides encoding svActRIIB-IgG Fc (E28W, S44T) (SEQ ID NO: 9) or ActRIIB-IgG Fc (E28W) (SEQ ID NO: 20) using a standard electroporation procedure. After transfection of the host cell line with the expression plasmids the cells were grown in serum-free selection medium without GHT for 2-3 weeks to allow for selection of the plasmid and recovery of the cells. Cells are selected until they achieved greater than 85% viability. This pool of transfected cells was then cultured in medium containing 150 nM methotrexate.

In a six-day expression assay, pools of svActRIIB-Fc (E28W, S44T) expressing cells showed higher cell titer, growth performance, and improved specific productivity (picogram/cell/day) of protein produced compared with pools of ActRIIB-Fc (E28W) expressing cells. Select pools, for example, produced about 1.2 g/liter for svActRIIB-Fc (E28W, S44T) compared with 0.9 g/liter for ActRIIB-Fc (E28W).

Each of an svActRIIB-Fc (E28W, S44T) and an ActRIIB-Fc (E28W) expressing cell line was scaled up using a typical fed-batch process. Cells were inoculated into a Wave bioreactor (Wave Biotech LLC). Cultures were fed three times with bolus feeds. 10 L were harvested on day 10, the remainder was harvested on day 11; both harvests underwent depth filtration followed by sterile filtration. The conditioned media was filtered through a 10 inch 0.45/0.2 micron pre filter, followed by a filtration through a 6 inch 0.2 micron filter.

### Protein Purification

Approximately 5 L of conditioned media was directly loaded onto a 220 mL MabSelect™ column Protein A column (GE Healthcare). The column was pre-equilibrated in PBS (phosphate-buffered saline: 2.67 mM potassium chloride, 138 mM sodium chloride, 1.47 mM potassium phosphate monobasic, 8.1 mM sodium phosphate dibasic, pH 7.4). The column was washed with the equilibration buffer until the reading at OD280 was approximately zero, and then the protein was eluted with 0.1M acetic acid.

The Mabselect™ Pool was applied to a 300 mL SP-HP column (GE Healthcare) (5×15 cm). The column was pre-equilibrated with 10mM NaOAC, pH 5. The column was then washed with the equilibration buffer until the reading at OD280 was approximately 0. The column was eluted with 20 column volumes of a gradient buffer from 0-150 mM NaCl in 10 mM NaOAC, pH 5. The SP-HP pool was concentrated, and filtered with a 0.2uM cellulose acetate (Corning) filter.
The sequences of the proteins used are set forth in the Table below.

| | | Linker- | |
|---|---|---|---|
| ActRIIB-Fc | ActRIIB sequence | Hinge | IgG2 Fc |
| svActRIIB- | ETRWCIYYNANWELERT | GGGGSV | APPVAGPSVFLFPPKPKDTLMISR |
| IgG₂Fc | NQTGLERCEGEQDKRLH | ECPPCP | TPEVTCVVVDVSHEDPEVQFNWY |
| (E28W, | CYASWRNSSGTIELVKKG | (SEQ ID | VDGVEVHNAKTKPREEQFNSTFR |
| S44T) | CWLDDFNCYDRQECVAT | NO: 27) | VVSVLTVVHQDWLNGKEYKCKV |
| (SEQ ID | EENPQVYFCCCEGNFCNE | | SNKGLPAPIEKTISKTKGQPREPQ |
| NO: 10) | RFTHLPEAGGPEVTYEPP | | VYTLPPSREEMTKNQVSLTCLVK |
| | PTAPT | | GFYPSDIAVEWESNGQPENNYKT |
| | (SEQ IDNO: 6) | | TPPMLDSDGSFFLYSKLTVDKSR |
| | | | WQQGNVFSCSVMHEALHNHYTQ |
| | | | KSLSLSPGK |
| | | | (SEQ ID NO: 22) |
| ActRIIB- | ETRWCIYYNANWELERT | GGGGSV | APPVAGPSVFLFPPKPKDTLMISR |
| IgG₂Fc | NQSGLERCEGEQDKRLH | ECPPCP | TPEVTCVVVDVSHEDPEVQFNWY |
| (E28W) | CYASWRNSSGTIELVKKG | (SEQ ID | VDGVEVHNAKTKPREEQFNSTFR |
| (SEQ ID | CWLDDFNCYDRQECVAT | NO: 27) | VVSVLTVVHQDWLNGKEYKCKV |
| NO: 21) | EENPQVYFCCCEGNFCNE | | SNKGLPAPIEKTISKTKGQPREPQ |
| | RFTHLPEAGGPEVTYEPP | | VYTLPPSREEMTKNQVSLTCLVK |
| | PTAPT | | GFYPSDIAVEWESNGQPENNYKT |
| | (SEQ ID NO: 19) | | TPPMLDSDGSFFLYSKLTVDKSR |
| | | | WQQGNVFSCSVMHEALHNHYTQ |
| | | | KSLSLSPGK |
| | | | (SEQ ID NO: 22) |

### Example 2

### Characterization of Polypeptides

Samples of the svActRIIB-Fc (E28W, S44T) (SEQ ID NO: 10) purified through the MabSelect™ step, and ActRIIB-Fc (E28W) (SEQ ID NO: 21) polypeptides purified through the SP-HP column step, as described above, were diluted with PBS, pH 7.4 to 0.2 mg/ml. The glycosylation profile of the polypeptides were then determined using SEC as described below.

Size exclusion chromatography (SEC). Experiments were performed on an Agilent 1100 HPLC system with two columns (TOSOHAAS G3000swxl, 7.8×300 mm) in tandem. 2× PBS was used as the mobile phase at 0.5 ml/minute.

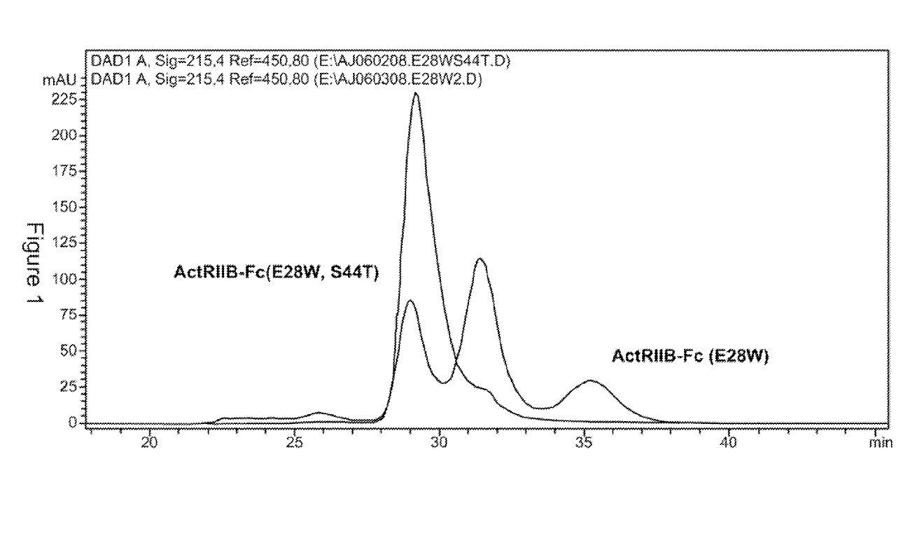

FIG. 1 shows a comparison between ActRIIB-Fc (E28W) and svActRIIB-Fc (E28W, S44T) on an SEC column using the protocols described above. svActRIIB-Fc (E28W, S44T) shows a single peak compared with ActRIIB-Fc (E28W), which shows three peaks. These correspond to the degree of N-linked glycosylation at the N42 position of the Fc dimers of both proteins. The single peak of the svActRIIB-Fc (E28W, S44T) polypeptide corresponds to fully glycosylated N-linked asparagines at position N42 of the dimer. The three peaks of ActRIIB-Fc (E28W) corresponds to (from left to right), fully glycosylated asparagines at N42, partially glycosylated asparagines at N42, and non-glycosylated asparagines at N42. Therefore, this demonstrates that the svActRIIB-Fc (E28W, S44T) molecule is fully glycosylated compared to ActRIIB-Fc (E28W), which is heterogeneous with respect to this glycosylation site, and thus more difficult to purify. In addition, preliminary studies indicate that the svActRIIB-Fc (E28W, S44T) molecule has addition improved manufacturability properties as set forth below. Additional studies also demonstrated that the least glycosylated peak of the ActRIIB-Fc (E28W) has lower physical and thermal stability than partially and fully glycosylated molecules.

Determination of K_{D} and IC₅₀ values of the receptor polypeptides for activin A, myostatin, and GDF-11 were obtained as described below.

### KinEx A™ Equilibrium Assays

Solution-based equilibrium-binding assays using KinExA™ technology (Sapidyne Instruments, Inc.) were used to determine the dissociation equilibrium (K_{D}) of ligand binding to ActRIIB-Fc polypeptides. UltraLink Biosupport beads (Pierce) was pre-coated with about 100 μg/m1 each of myostatin, GDF-11, and activin A overnight, and then blocked with BSA. 1 pM and 3 pM of ActRIIB-Fc (E28W) (SEQ ID NO: 21) and svActRIIB-Fc (E28W, S44T) (SEQ ID NO: 10) samples were incubated with various concentrations (0.7 fM to 160 pM) of myostatin, activin A, and GDF-11 respectively in sample buffer at room temperature for 8 hours before being run through the ligand-coated beads. The amount of the bead-bound soluble receptor was quantified by fluorescent (Cy5) labeled goat anti-human-Fc antibody at 1 mg/ml in superblock. The binding signal is proportional to the concentration of free soluble receptor at equilibrium with a given myostatin, activin A, or GDF-11 concentration. K_{D} was obtained from the nonlinear regression of the competition curves using a dual-curve one-site homogeneous binding model provided in the KinEx A™ software (Sapidyne Instruments, Inc.). The K_{D} values obtained for each are given in the table below.

| | Myostatin | GDF-11 | Activin A |
|---|---|---|---|
| ActRIIB-Fc (E28W) | 0.1 pM | 0.1 pM | 0.2 pM |
| svActRIIB-Fc (E28W, | 0.1 pM | 0.1 pM | 0.1 pM |
| S44T) | | | |

### C2C12 Cell Based Activity Assay

The ability of ActRIIB-Fc (E28W) (SEQ ID NO: 21) and svActRIIB-Fc (E28W, S44T) (SEQ ID NO: 10) to inhibit the binding of activin A, GDF-11, or myostatin to the wild type activin IIB receptor-Fc was tested using a cell based activity assay as described below.

A myostatin/activin/GDF-11-responsive reporter cell line was generated by transfection of C2C12 myoblast cells (ATCC No: CRL-1772) with a pMARE-luc construct. The pMARE-luc construct is made by cloning twelve repeats of the CAGA sequence, representing the myostatin/activin response elements (Dennler et al. EMBO 17: 3091-3100 (1998)) into a pLuc-MCS reporter vector (Stratagene cat #219087) upstream of the TATA box. The C2C12 cells naturally express activin receptor IIB on their cell surface. When myostatin/activinA/GDF-11 binds the cell receptors, the Smad pathway is activated, and phosphorylated Smad binds to the response element (Macias-Silva et al. Cell 87:1215 (1996)), resulting in the expression of the luciferase gene. Luciferase activity was then measured using a commercial luciferase reporter assay kit (cat #E4550, Promega, Madison, Wis.) according to manufacturer's protocol. A stable line of C2C12 cells that has been transfected with pMARE-luc (C2C12/pMARE) was used to measure activity according to the following procedure. Reporter cells were plated into 96 well cultures. Screening using dilutions of the ActRIIB-IgG2 Fc fusions constructed as described above was performed with the concentration fixed at 4 nM activin A, myostatin, and GDF-11. Each of these ligands was pre-incubated with the receptors at several concentrations. Activity was measured by determining the luciferase activity in the treated cultures. The IC₅₀ values were determined for each polypeptide. These are shown in the Table below. These values are given in Table below.

| | Myostatin | GDF-11 | Activin A |
|---|---|---|---|
| ActRIIB-Fc (E28W) | 0.95 nM | 2.4 nM | 3.2 nM |
| svActRIIB-Fc (E28W, | 1.07 nM | 2.4 nM | 3.6 nM |
| S44T) | | | |

Thus the cell based activities are approximately the same for ActRIIB-Fc (E28W) and svActRIIB-Fc (E28W, S44T).

### Stability at Low pH

Stability of a protein at low pH is a useful parameter in considering the manufacturability of the protein, since the viral inactivation step of a commercial production process typically is carried out at low pH, such as between about pH 3.0 to 4.0.

To assess the short term protein stability effects at low pH experienced during the viral inactivation step of commercial protein purification the following test was performed. Each protein was diluted to 10 mg/ml of 100 mM sodium acetate, pH 3.5. This was stored at 25° C. and analyzed at time 0, at 2 hours and at 24 hours using SEC analysis. SEC analysis was performed as described above, and percentage of high molecular weight aggregates was determined.

% HMW aggregate

| | T = 0 | T = 2 hours | T = 4 hours |
|---|---|---|---|
| ActRIIB-Fc (E28W) | 1.53 | 1.36 | 13.74 |
| svActRIIB-Fc | 1.66 | 2.17 | 8.93 |
| (E28W, S44T) | | | |

Thus the percentage of high molecular weight aggregates produced at pH 3.5 is substantially less for svActRIIB-Fc (E28W, S44T) than ActRIIB-Fc (E28W) at 4 hours.

Additional studies showed that svActRIIB-Fc (E28W, S44T) showed better reversibility than ActRIIB-Fc (E28W) from exposure to pH 3.0, 3.5 and 5.0, and that svActRIIB-Fc (E28, S44T) was more homogeneous that ActRIIB-Fc (E28W) at all pHs.

Thus, the svActRIIB-Fc (E28W, S44T) polypeptides are demonstrated to have improved manufacturability characteristics, in particular, improved stability at low pH, and greater homogeneity at all pHs compared with ActRIIB-Fc (E28W) while retaining the ability to inhibit activin A, myostatin, and GDF-11 activity.

### Example 3

### Determination of In Vivo Efficacy

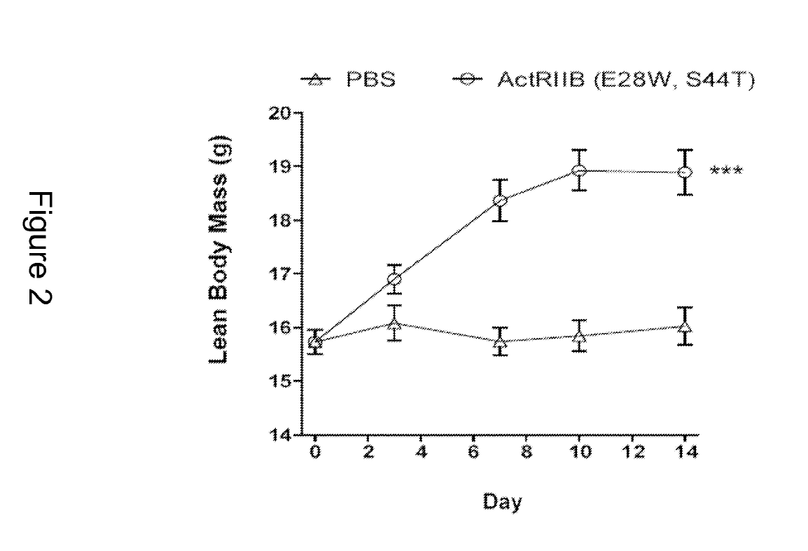

11-week-old female C57B1/6 mice were purchased from Charles River Laboratories. The mice (ten mice per group) were administered a single dose (10 mg/kg) of svActRIIB-Fc (E28W, S44T) (SEQ ID NO: 10) or vehicle (PBS). Lean body mass was determined by NMR (PIXImus, GE LUNAR Corporation) at 3, 7, 10 and 14 days after dose administration for the ten animals in each group. The results for each set of mice are shown in FIG. 2. It can be seen that a single dose of svActRIIB-Fc (E28W, S44T) significantly increased lean body mass in the animals. (P<0.001, based on repeated measurement ANOVA. n=10 animals per group).

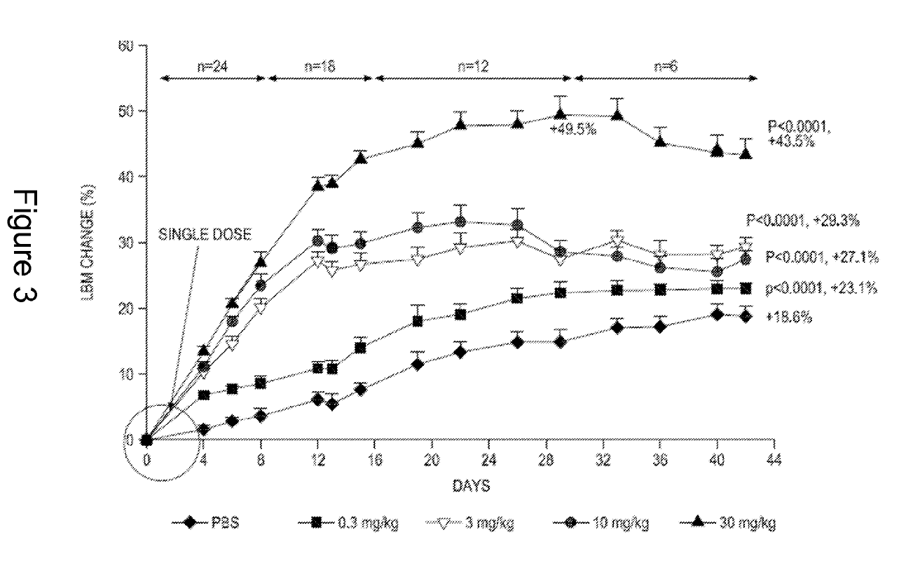

A study to determine dose-response efficacy was carried out as follows. Escalating single doses of 0, 0.3, 3, 10, and 30 mg/kg of svActRIIB-Fc (E28W, S44T) (SEQ ID NO: 10) in PBS was administered subcutaneously to female 10-12 week old C57B1/6 mice (Charles River Laboratories). Six animals were initially in each dosage group including the PBS control group. Lean body mass was determined by NMR (PIXImus, GE LUNAR Corporation) every two to four days for the forty-two days of the study. At the end of each week, one animal from each group was sacrificed to obtain additional data (six in total each week from all six groups), and the lean body mass determined for the remaining animals in subsequent weeks. The results are set out in FIG. 3. It can be seen that the svActRIIB-Fc (E28W, S44T) polypeptide at all doses significantly increased muscle mass in the animals, in a dose-dependent manner.

In further studies, head to head comparisons between ActRIIB-Fc (E28W) (SEQ ID NO: 21) and svActRIIB-Fc (E28W, S44T) (SEQ ID NO: 10) were performed on female C57B1/6 mice (Charles River Laboratories, 10 animals per group) to measure the increase in lean muscle mass and body weight changes after a single dose of 10 mg/kg of each soluble receptor compared with a control group (administered PBS). Lean body mass was determined by NMR (PIXImus, GE LUNAR Corporation), and body weight change was determined by weighing the animals periodically for 37 days. The results at the end of this comparative study was that ActRIIB-Fc (E28W) (SEQ ID NO: 21) showed an increase of 24% in lean muscle mass and 25% in increase of body weight compared with an increase of 25% in lean muscle mass and 20% increase in body weight for svActRIIB-Fc (E28W, S44T) (SEQ ID NO: 10), compared with an increase of 5% lean muscle mass and 9% increase body weight for the control group.

Therefore, it can be seen that svActRIIB-Fc (E28W, S44T) retains comparable in vivo efficacy compared with ActRIIB-Fc (E28W) while having improved manufacturability characteristics.

Further examples of in vivo testing of ActRIIB-Fc (E28W) can be found in U.S. Pat. No. 7,947,646. For example, Example 3 of U.S. Pat. No. 7,947,646, demonstrates use of ActRIIB-Fc (E28W) in the treatment or prevention of various diseases or conditions including muscle wasting, cancer (colon-26, ovarian, testicular, and adrenal cancers), anorexia, hindlimb suspension, hypogonadism, and osteoporesis. Example 3 of U.S. Pat. No. 7,947,646 is herein incorporated by reference, in its entirety, for this express purpose. Further in vivo examples can be found in PCT/US2014/14490 and PCT/US2012/070571, each of which is herein incorporated by reference, in its entirety, for this express purpose.

### Example 4

### Improved Manufacturability with Modified Hinge Linkers

Additional linkers and modified hinge regions were constructed to test for further improvement of protein expression and manufacturability of the stabilized ActRIIB (E28W, S44T) polypeptides. Modified linker/hinge sequences based on modifications of hinge linker #1 were generated using overlap extension PRC mutagenesis methods, according to Mikaelian et al., Methods in Molecular Biology, 57, 193-202 (1996), and well known methodology.

The modified hinge linkers designed to perform well with IgG2 Fc fusions are hinge linker #2-7 set forth below (in comparison to hinge linker #1 sequences).

hinge linker #1 polynucleotide
(SEQ ID NO: 26)
ggagggggaggatctgtcgagtgcccaccgtgccca.
hinge linker #1 polypeptide
(SEQ ID NO: 27)
GGGGSVECPPCP
hinge linker #2 polynucleotide
(SEQ ID NO: 37)
ggagggggaggatctgagcgcaaatgttgtgtcgagtgcccaccgtgc
hinge linker #2 peptide
(SEQ ID NO: 38)
GGGGSERKCCVECPPC
hinge linker #3 polynucleotide
(SEQ ID NO: 39)
ggagggggaggatctggtggaggtggttcaggtccaccgtgc
hinge linker #3 peptide
(SEQ ID NO: 40)
GGGGSGGGGSGPPC
hinge linker #4 polynucleotide
(SEQ ID NO: 41)
ggagggggaggatctggtggaggtggttcaggtccaccggga
hinge linker #4 peptide
(SEQ ID NO: 42)
GGGGSGGGGSGPPG
hinge linker #5 polynucleotide
(SEQ ID NO: 43)
ggagggggaggatctgagcgcaaatgtccaccttgtgtcgagtgcccacc
gtgc
hinge linker #5 peptide
(SEQ ID NO: 44)
GGGGSERKCPPCVECPPC
hinge linker #6 peptide
(SEQ ID NO: 45)
GPASGGPASGPPCP
hinge linker #7 peptide
(SEQ ID NO: 46)
GPASGGPASGCPPCVECPPCP

The following hinge linkers #8 to #10 below were designed to perform well with an IgG1Fc (SEQ ID NO: 23) or the modified IgG1Fc given below (SEQ ID NO: 47 below).

modified IgG1 Fc
(SEQ ID NO: 47)
APELLGGPSVFLFPPKPKDTLMISRTPEVTCVVVDVSHEDPEVKFNWYVD
GVEV
HNAKTKPREEQYNSTYRVVSVLTVLHQDWLNGKEYKCKVSNKALPAPIEK
TISKAKGQP
REPQVYTEPPSREEMTKNQVSETCLVKGFYPSDIAVEWESNGQPENN
YKTTPPVLDSDGSFFLYSKLTVDKSRWQQGNVFSCSVMHEALHNHYTQKS
LSLSPGK
hinge linker #8 peptide
(SEQ ID NO: 48)
GGGGSVDKTHTCPPCP
hinge linker #9 peptide
(SEQ ID NO: 49)
GGGGSVDKTHTGPPCP
hinge linker #10 peptide
(SEQ ID NO: 50)
GGGGSGGGGSVDKTHTGPPCP

Testing of modified hinge linker sequences with svActRIIB-Fc (28W, S44T) was performed as follows. Polynucleotides encoding svActRIIB (E28W, S44T) (SEQ ID NO: 5), polynucleotides encoding the modified hinge linkers shown above, and polynucleotides encoding IgG2 Fc (SEQ ID NO: 22) or polynucleotides encoding IgG1 Fc (SEQ ID NO: 23) or modified IgG1 Fc (SEQ ID NO: 47) were subcloned into vectors as described in Example 1 and expressed using the transient 293-6E expression system as described in Example 1, except for the following changes: F17 media (Invitrogen) supplemented with 1.1 mg/ml Pluronic, 6 mM L-glutamine and 25 μg/ml geneticin (Invitrogen) was used in place of Freestyle 293 medium as described in Durocher et al., Nucleic Acids Research 30, No. 3, e9 (2002)). The cultures were grown for seven days at 37° C. after transfection. Aliquots were centrifuged to remove cells, and the supernatant was mixed with loading buffer before being heated and loaded onto a 4-20% tris-glycine gel for analysis by Western Blot. After the protein was transferred to a nitrocellulose membrane, samples were probed with a hydrogen peroxidase conjugated anti-human Fc antibody (Pierce #31423) at a dilution of 1:1000.

Protein purification was performed using the following procedure. Approximately 0.25 L of the conditioned media containing the svActRIIB-Fc variants were concentrated using a 5 ft² 10K membrane tangential flow filter. The concentrated material was applied to a 5 mL Protein A High Performance ColumnTM (GE Heathcare) which had been equilibrated with PBS (Dulbecco's with no magnesium chloride or calcium chloride). After washing the column with the equilibration buffer until the absorbance at 280 nm (OD₂₈₀) was less than 0.1, the bound protein was eluted with 0.1 M glycine-HCl, pH 2.7, and immediately neutralized with 1 M Tris-HCl, pH 8.5.

The portion of aggregate in percent and the portion of half molecule in percent were determined by the following method. Denaturing size exclusion chromatography experiments were performed by injecting a 50 μl aliquot of each sample onto an HPLC system with two size exclusion columns (TOSOHAAS G3000swxl) in tandem. The mobile phase contains 5 M GuHCl in phosphate buffered saline (PBS). All samples were diluted to 1 mg/mL in PBS with 7 M GuHCl. The portion of aggregate in percent is determined from the total peak areas of the peaks eluted before the main peak, whereas the portion of half-molecule in percent is determined from the total peak areas of the peaks eluted after the main peak. The half-molecule are believed to represent inactive half-molecules.

Aggregate and half-molecule distribution of svActRIIB-Fc (E28W, S44T) with the various hinge linkers are set forth in the following table.

| Hinge linker | % | % half |
|---|---|---|
| sequence | aggregate | molecule |
| GGGGSVECPPC | 0.63 | 15.12 |
| (SEQ ID NO: 27) | | |
| GGGGSERKCCVECPPC | 15.01 | 7.19 |
| (SEQ ID NO: 38) | | |
| GGGGSGGGGSGPPC | 0.56 | 3.83 |
| (SEQ ID NO: 40) | | |
| GGGGSGGGGSGPPG | 0.00 | 99.03 |
| (SEQ ID NO: 42) | | |
| GGGGSERKCPPCVECPPC | 1.09 | 3.81 |
| (SEQ ID NO: 44) | | |

Thus certain linkers may improve manufacturability of the stabilized ActRIIB-Fc (E28W, S44T) according to these preliminary tests by reducing the percentage of inactive half-molecules produced.

### Example 5

Prediction of Response to Therapy with Activin Inhibitors Using a Biomarker of Autocrine Activin Signaling.

Background:

Activins are distinct members of the TGF-β family that play important roles in growth and differentiation of normal tissues and in regulating tumor growth. Activin A signals are transduced by the high affinity activin type 2B receptor (ActR2B), leading to downstream Smad signaling and modulation of gene transcription. Recent observations that show the activin A mRNA precursor INHBA and its target downstream genes such as COL11A1 are expressed in the ovarian intra- and peri-tumoral stroma, and not in the epithelial tumor cells (Cheon 2013, Tothill 2008).

Stromal activin A overexpression is a component of a metastasis-associated gene expression signature that predicts short survival across ovarian, colon, gastric, and breast cancers, non-small cell lung cancers and neuroblastoma.

Studies of additional solid tumors have confirmed an important role of the stromal activin A epithelial cancer activin receptor interaction beyond ovarian cancer. The INHBA activin A mRNA precursor was upregulated 6.5-fold in infiltrating ductal carcinomas as compared with benign breast tissue, and patients with high levels of activin A were more likely to have metastases to bone. A study of 66 patients with Stage 1 lung adenocarcinomas showed that those with elevated INHBA mRNA had shorter median survival of 60 months compared with 136 months (p=0.008) for those without INHBA elevation. INHBA is part of the Oncotype DX 12 gene colon cancer recurrence score assay (Genomic Health, Redwood City, Calif.), validated to predict colon cancer recurrence in a large scale prospective study of n=892 subjects who had participated in the NSABP C-07 trial (Yothers 2013). Studies of Asian patients with gastric cancer have found INHBA upregulation (Wu 2005); in one study, INHBA was the most upregulated gene among the 17,000 genes on the microarray platform (19.8 fold tumor:normal) (Yang 2007); INHBA upregulation in Asian gastric tumors was confirmed by a second group . Expression of INHBA in Asian gastric tumors (n=132) associated with short disease-free survival. In prostate cancer, high levels of activin A correlated with elevated prostate-specific antigen (PSA) (p=0.0001), high Gleason score (p=0.011), and increased metastasis to bone. In glioma, INHBA elevation and the mesenchymal gene expression signature correlated with shorter recurrence free survival (Cheng 2012).

The RNAscope assay from ACD Bio now allows simultaneous detection of two mRNAs at single cell anatomic resolution (Shames 2013). Likewise, antibodies can be used to characterize two proteins at single cell anatomic resolution (Abalsamo 2012).

Description:

In an embodiment, a ligand-receptor coexpression biomarker is the co-expression of the activin A ligand and its cognate high affinity receptor, the activin type IIB receptor, together at the anatomic location where tumor invasion into surrounding tissue occurs. High levels of the ligand-receptor coexpression biomarker can predict for clinical responses to anti-activin therapeutics including antibodies/peptibodies/soluble receptors that neutralize Activin A, B, C etc, antibodies that neutralize the activin type IIB receptor, etc. Examples therapeutics includes SEQ ID NOs 10 and 21 disclosed herein (e.g., Example 1).

In one embodiment, the ligand-receptor coexpression biomarker is detected at the RNA level using the RNAscope assay (INHBA mRNA probes and ACVR2B mRNA probes). The probes can be designed to a region of the mRNA that is conserved across mouse and human species so that the same test can identify the ligand-receptor coexpression biomarker for use in preclinical mouse as well as clinical human tumors. In some aspects, the probes are designed to hybridize to exon:exon junctions in one or both of the INHBA and ACVR2B mRNAs.

In some aspects, the sequences of human INHBA can be found on the NCBI website on Jan. 6, 2014 at RefSeq: NM_002192. In some aspects, the sequences of human ACVR2B can be found on the NCBI website on Jan. 6, 2014 at RefSeq: NM_001106. One or more probes capable of binding each of these targets are generally known in the art. The probe or probes can be of any length capable of functioning as a probe specific for the respective target, e.g., 10, 15, 20, 25, 30, 35, 40, 45, or 50 bases in length (or any integer in between).

In another embodiment, the ligand-receptor coexpression biomarker is detected at the protein level using specific monoclonal antibodies for the activin A ligand and for the Activin type IIB receptor; one set of antibodies can recognize mouse Activin A and ACTR2B, and another set of antibodies can recognize human Activin A and ACTR2B. One or more antibodies capable of binding each of these targets are generally known in the art.

Biomarker-based selection of certain patients for treatment can lead to higher drug response rates, longer duration of treatment, time until progression and improved survival as compared to treatment of a general patient population without the use of the ligand-receptor coexpression biomarker.

In some aspects, ligand-receptor coexpression biomarker-based testing for ligand or receptor can be performed without regard to anatomic location.
References for Example 5:
Abalsamo L, Spadaro F, Bozzuto G, Paris L, Cecchetti S, Lugini L, et al. Inhibition of phosphatidylcholine-specific phospholipase c results in loss of mesenchymal traits in metastatic breast cancer cells. Breast cancer research: BCR. 2012; 14 (2):R50.
Cheng W Y, Kandel J J, Yamashiro D J, Canoll P, Anastassiou D. A multi-cancer mesenchymal transition gene expression signature is associated with prolonged time to recurrence in glioblastoma. PloS one. 2012; 7 (4):e34705.
Cheon D J, Tong Y, Sim M S, Dering J, Berel D, Cui X, et al. A collagen-remodeling gene signature regulated by tgfbeta signaling is associated with metastasis and poor survival in serous ovarian cancer. Clinical cancer research : an official journal of the American Association for Cancer Research. 2013.
Shames D S, Carbon J, Walter K, Jubb A M, Kozlowski C, Januario T, et al. High heregulin expression is associated with activated her3 and may define an actionable biomarker in patients with squamous cell carcinomas of the head and neck. PloS one. 2013; 8 (2):e56765.
Tothill R W, Tinker A V, George J, Brown R, Fox S B, Lade S, et al. Novel molecular subtypes of serous and endometrioid ovarian cancer linked to clinical outcome. Clinical cancer research: an official journal of the American Association for Cancer Research. 2008; 14 (16):5198-208.
Wu M S, Lin Y S, Chang Y T, Shun C T, Lin M T, Lin J T. Gene expression profiling of gastric cancer by microarray combined with laser capture microdissection. World journal of gastroenterology: WJG. 2005; 11 (47):7405-12.
Yang S, Shin J, Park K H, Jeung H C, Rha S Y, Noh S H, et al. Molecular basis of the differences between normal and tumor tissues of gastric cancer. Biochimica et biophysica acta. 2007; 1772 (9):1033-40.
Yothers G, O'Connell M J, Lee M, Lopatin M, Clark-Langone K M, Millward C, et al. Validation of the 12-gene colon cancer recurrence score in nsabp c-07 as a predictor of recurrence in patients with stage ii and iii colon cancer treated with fluorouracil and leucovorin (fully) and fully plus oxaliplatin. Journal of clinical oncology : official journal of the American Society of Clinical Oncology. 2013.

### Example 6

Chromogenic 2-Plex RNASCOPE® ISH Assay Development and Validation of Human INHBA and ACVR2B Target Genes in One FFPE Xenograft TMAS.

Objective:

Develop and demonstrate validation of Chromogenic 2-plex RNAscope In Situ Hybridization (ISH) assay for detection of Human Activin A and Activin A receptor type IIB.

Background:

The RNAscope® 2.0 HD Assay is an RNA in situ hybridization (ISH) technology with a unique probe design strategy that allows simultaneous signal amplification and background suppression to achieve single-molecule visualization while preserving tissue morphology. RNAscope is compatible with routine formalin-fixed, paraffin-embedded tissue specimens and can use either conventional chromogenic dyes for bright-field microscopy or fluorescent dyes for multiplex analysis. Tissues and cells mounted on slides are first pretreated (antigen retrieval) to prepare for hybridization. Oligonucleotide target probes are hybridized to the RNA in the sample, followed by a series of steps and washes designed to amplify the signal. The signal on the target RNA molecule is detected by using a chromogenic substrate reaction which produces a precipitate visible under a common bright-field microscope at 10-20× magnification as a distinct brown or red dot. The number of dots observed in each cell depends on the tissue type and target.

Project Scope:

Phase I: Sample Validation and Optimization
Evaluate sample quality using positive and negative reference controls

Phase II: Marker Evaluation and Data Analysis
Perform RNAscope assay on qualified samples using target probe, collect images, score and analyze data

Protocols, Materials, and Methods:

Protocol

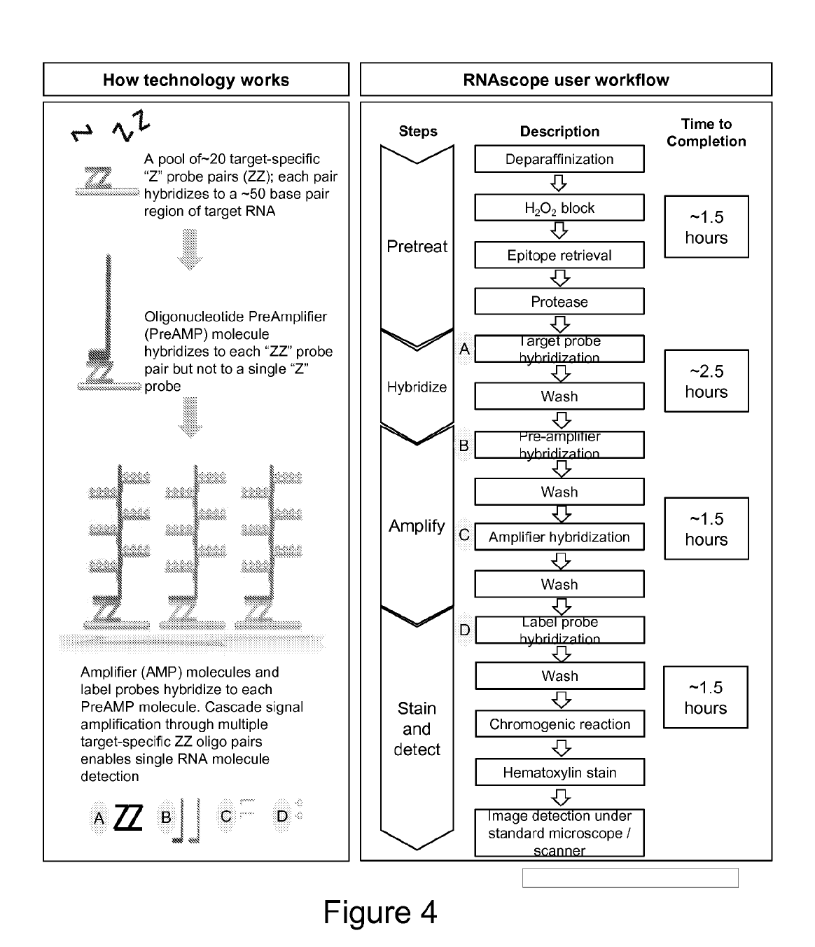

An overview of a typical RNAscope® technology workflow is shown in FIG. 4.

Based on the assessment of tissue samples with reference positive and negative control probes, optimal pretreatment conditions for the sample tissues were established to maximize signal-to-noise ratio.

Standard RNAscope 2plex Assay protocol conditions were used for the analysis of study samples with no modification in pretreatment conditions. See www.acdbio.com website as of Jul. 10, 2014 for protocol and user manual. Note: The conditions determined to be optimal for the study samples will likely be suitable for all future studies of these samples, as well as for the same tissue type prepared under very similar conditions.

Materials and Methods

RNA in situ hybridization for Hs-INHBA and Hs-ACVR2B mRNA was performed manually using RNAscope® 2plex FFPE Reagent Kit (Advanced Cell Diagnostics, Inc., Hayward, Calif.) according to the manufacturer's instructions. Briefly, 5 μm formalin fixed, paraffin embedded (FFPE) tissue sections were pretreated with heat and protease prior to hybridization with the target oligo probes. Preamplifier, amplifier and HRP- or AP-labeled oligos were then hybridized sequentially, followed by chromogenic precipitate development. Each sample was quality controlled for RNA integrity with a RNAscope probe specific to PPIB RNA and for background with a probe specific to bacterial dapB RNA. Specific RNA staining signal was identified as red or green punctate dots. Samples were counterstained with Gill's Hematoxylin.

Brightfield images were acquired using a Zeiss Axio Imager M1 upright microscope with AxioVisionLE software. Images were acquired with a 40× objective.

Samples and Probes
Sample Source: One (1) TMA sample with 8 cores.
RNAscope® Probes
   Homo sapiens inhibin beta A (INHBA) mRNA
      Probe symbol: Hs-INHBA-C2 (Cat. No. 415111-C2)
      Probe GenBank Accession Number: NM_002192.2
      Number of double Z probe pairs: 20
      Gene region probes designed against: 364-1374 nt
   Homo sapiens activin A receptor type IIB (ACVR2B) mRNA
      Probe symbol: Hs-ACVR2B (Cat. No. 415121)
      Probe GenBank Accession Number: NM_001106.3
      Number of double Z probe pairs: 20
      Gene region probes designed against: 627-1503 nt
   ACD 2-plex positive control probe for human
      Probe symbol: Hs-PolR2A-C1/Hs-PPIB-C2 (Cat. No. 320541)
      Probe name: Polymerase (RNA) II (DNA directed) polypeptide A
      Probe name: Peptidylprolyl Isomerase B (Cyclophilin B)
   ACD 2-plex negative control probe
      Probe symbol: dapB-C1/dapB-C2 (Cat. 320751)
      Probe name: B. subtilis gene dihydrodipicolinate reductase

| SEQ | | |
|---|---|---|
| ID | | |
| NO | DESCRIPTION | SEQUENCE |
| 51 | NM_002192.2; | agtacagtataaaacttcacagtgccaataccatgaagaggagctcaga |
| | CDS is position | cagctcttaccacatgatacaagagccggctggtggaagagtggggacc |
| | 248-1528 | agaaagagaatttgctgaagaggagaaggaaaaaaaaaacaccaaaaaa |
| | | aaaaataaaaaaatccacacacacaaaaaaacctgcgcgtgagggggga |
| | | ggaaaagcagggccttttaaaaaggcaatcacaacaacttttgctgcca |
| | | ggatgcccttgctttggctgagaggatttctgttggcaagttgctggat |
| | | tatagtgaggagttcccccaccccaggatccgaggggcacagcgcggcc |
| | | cccgactgtccgtcctgtgcgctggccgccctcccaaaggatgtaccca |
| | | actctcagccagagatggtggaggccgtcaagaagcacattttaaacat |
| | | gctgcacttgaagaagagacccgatgtcacccagccggtacccaaggcg |
| | | gcgcttctgaacgcgatcagaaagcttcatgtgggcaaagtcggggaga |
| | | acgggtatgtggagatagaggatgacattggaaggagggcagaaatgaa |
| | | tgaacttatggagcagacctcggagatcatcacgtttgccgagtcagga |
| | | acagccaggaagacgctgcacttcgagatttccaaggaaggcagtgacc |
| | | tgtcagtggtggagcgtgcagaagtctggctcttcctaaaagtccccaa |
| | | ggccaacaggaccaggaccaaagtcaccatccgcctcttccagcagcag |
| | | aagcacccgcagggcagcttggacacaggggaagaggccgaggaagtgg |
| | | gcttaaagggggagaggagtgaactgttgctctctgaaaaagtagtaga |
| | | cgctcggaagagcacctggcatgtcttccctgtctccagcagcatccag |
| | | cggttgctggaccagggcaagagctccctggacgttcggattgcctgtg |
| | | agcagtgccaggagagtggcgccagcttggttctcctgggcaagaagaa |
| | | gaagaaagaagaggagggggaagggaaaaagaagggcggaggtgaaggt |
| | | ggggcaggagcagatgaggaaaaggagcagtcgcacagacctttcctca |
| | | tgctgcaggcccggcagtctgaagaccaccctcatcgccggcgtcggcg |
| | | gggcttggagtgtgatggcaaggtcaacatctgctgtaagaaacagttc |
| | | tttgtcagtttcaaggacatcggctggaatgactggatcattgctccct |
| | | ctggctatcatgccaactactgcgagggtgagtgcccgagccatatagc |
| | | aggcacgtccgggtcctcactgtccttccactcaacagtcatcaaccac |
| | | taccgcatgcggggccatagcccctttgccaacctcaaatcgtgctgtg |
| | | tgcccaccaagctgagacccatgtccatgttgtactatgatgatggtca |
| | | aaacatcatcaaaaaggacattcagaacatgatcqtggaqgagtgtggg |
| | | tgctcatagagttgcccagcccagggggaaagggagcaagagttgtcca |
| | | gagaagacagtggcaaaatgaagaaatttttaaggtttctgagttaacc |
| | | agaaaaatagaaattaaaaacaaaacaaaaaaaaaaacaaaaaaaaaca |
| | | aaagtaaattaaaaacaaaacctgatgaaacagatgaaggaagatgtgg |
| | | aaaaaatccttagccagggctcagagatgaagcagtgaaagagacagga |
| | | attgggagggaaagggagaatqqtgtaccctttatttcttctgaaatca |
| | | cactgatgacatcagttgtttaaacggggtattgtcctttccccccttg |
| | | aggttcccttgtgagccttgaatcaaccaatctagtctgcagtagtgtg |
| | | gactagaacaacccaaatagcatctagaaagccatgagtttgaaagggc |
| | | ccatcacaggcactttcctacccaattacccaggtcataaqqtatgtct |
| | | gtgtgacacttatctctgtgtatatcagcatacacacacacacacacac |
| | | acacacacacacacacaggcatttccacacattacatatatacacatac |
| | | tggtaaaagaacaatcgtgtgcaggtggtcacacttcctttttctgtac |
| | | cacttttgcaacaaaacaa |
| 52 | NM_001106.3; | gtgcgcggggcggcgccgcggaacatgacggcgccctgggtggccctcg |
| | CDS is positions | ccctcctctggggatcgctgtgcgccggctctgggcgtggggaggctga |
| | 25-1563 | gacacgggagtgcatctactacaacgccaactgggagctggagcgcacc |
| | | aaccagagcggcctggagcgctgcgaaggcgagcaggacaagcggctgc |
| | | actgctacgcctcctggcgcaacagctctggcaccatcgagctcgtgaa |
| | | gaagggctgctggctagatgacttcaactgctacgataggcaggagtgt |
| | | gtggccactgaggagaacccccaggtgtacttctgctgctgtgaaggca |
| | | acttctgcaacgaacgcttcactcatttgccagaggctgggggcccgga |
| | | agtcacgtacgagccacccccgacagcccccaccctgctcacggtgctg |
| | | gcctactcactgctgcccatcgggggcctttccctcatcgtcctgctgg |
| | | ccttttggatgtaccggcatcgcaagcccccctacggtcatgtggacat |
| | | ccatgaggaccctgggcctccaccaccatcccctctggtgggcctgaag |
| | | ccactgcagctgctggagatcaaggctcgggggcgctttggctgtgtct |
| | | ggaaggcccagctcatgaatgactttgtagctgtcaagatcttcccact |
| | | ccaggacaagcagtcgtggcagagtgaacgggagatcttcagcacacct |
| | | ggcatgaagcacgagaacctgctacagttcattgctgccgagaagcgag |
| | | gctccaacctcgaagtagagctgtggctcatcacggccttccatgacaa |
| | | gggctccctcacggattacctcaaggggaacatcatcacatggaacgaa |
| | | ctgtgtcatgtagcagagacgatgtcacgaggcctctcatacctgcatg |
| | | aggatgtgccctggtgccgtggcgagggccacaagccgtctattgccca |
| | | cagggactttaaaagtaagaatgtattgctgaagagcgacctcacagcc |
| | | gtgctggctgactttggcttggctgttcgatttgagccagggaaacctc |
| | | caggggacacccacggacaggtaggcacgagacggtacatggctcctga |
| | | ggtgctcgagggagccatcaacttccagagagatgccttcctgcgcatt |
| | | gacatgtatgccatggggttggtgctgtgggagcttgtgtctcgctgca |
| | | aggctgcagacggacccgtggatgagtacatgctgccctttgaggaaga |
| | | gattggccagcacccttcgttggaggagctgcaggaggtggtggtgcac |
| | | aagaagatgaggcccaccattaaagatcactggttgaaacacccgggcc |
| | | tggcccagctttgtgtgaccatcgaggagtgctgggaccatgatgcaga |
| | | ggctcgcttgtccgcgggctgtgtggaggagcgggtgtccctgattcgg |
| | | aggtcggtcaacggcactacctcggactgtctcgtttccctggtgacct |
| | | ctgtcaccaatgtggacctgccccctaaagagtcaagcatctaagccca |
| | | ggacatgagtgtctgtccagactcagtggatctgaagaaaaaaggaaaa |
| | | aaagttgtgttttgttttggaaatcccataaaaccaacaaacacataaa |
| | | atgcagctgctattttaccttgactttttattattattattataattat |
| | | tataattattattattaatattattttttggattggatcagtttttacc |
| | | agcatattgctctactgtatcacaaacagcggacacgtcagcaggcgtt |
| | | gaggtgctgagctgtggatgcagaaccagcgccatgctgaagagcctca |
| | | gccacctcctgtcctttgggattcgtttttcccgctttctctttgtttg |
| | | tcgtctcagaatctgtgacacaaagaaacccatctcctgtcttaggaaa |
| | | cctaatgctgcaaactctacctagaggaacctttgaagactgttacata |
| | | agaacataccttcctcagaagaggagtttcctctgccctctgcccttct |
| | | cccctgcctccctccctcccctccttttattttgttttagtgagcttaa |
| | | gaaacagcagatgtgtctttcacggatctaacgggtgttgtcctgatcg |
| | | agaaaaaaactgggatgagaatggtttggactggagttggaaggggagg |
| | | acggtactgggggtagggtttggaacagagctacactggactcgggcac |
| | | attcggagcagcatcctttagtatggaggctacttctcaggtaaccagg |
| | | aattgaggggaaggaccttgtggaggccgagcattaacagcaagagcgg |
| | | ggtttggagaaagtctgagattgggtgcagccctgacttacctgctggc |
| | | cctgaccagtttcttttcactaacttggccttgggcataggatgaaaca |
| | | ttttttctgccctaattttaaaactaggtgagggtagaatcatcacagg |
| | | ttaggaatacattcttcataagacacgatgctgtaaatacccttaatgg |
| | | acgaaaagttgaaatacttttgtttcctcttggagcagttcagggaaat |
| | | gcccacaggggattgtcctgcacagatagggcaagaggatttcctgggt |
| | | ggagtctgccaaggcctgcctcgctggggaccccagagtcctgcacctc |
| | | tggttccgccccaggtggtgacattactgtccccgttctgtggctcgtg |
| | | gacaagactttctccagaccccttaaagtggtacatattctaaaaaact |
| | | gtttttctattatgccataaccttgctctagtcagtgaatgttcctaat |
| | | gctgctgtttcaacatttgaattctttttaatttatgaaacatgctaaa |
| | | ttttttttttcaaacaaaacacacacatccacatatacacatgcttcgc |
| | | tatgtggcttccaaggtttaaattttgaaaagtaaaagaattaaaactt |
| | | cacgaccacagatcacctcaaaccagaaatacctcagaattttctactt |
| | | atgtaaggtttattatatattttgttagttgtgttgtcttgtagtaagt |
| | | atattttaatgtaagttggcttttgtgacaaggaagtttaaaagaaata |
| | | gagaaaaagaaaaaagtttgcatcttctagggagtgctaccatttttgt |
| | | ttgataacgcccccttgtaaataattgtcatcaactgtaggttggctgt |
| | | ctgggccaagtctgggcatttatcagtcttgtttgtgaaggcttttcct |
| | | tctggtttctttagatcattttatttaaaaacagtgcatctcttcatcg |
| | | tgagggtaggcaaggcgggggccgtggggagaggttgacctgggtgaga |
| | | actgaagaggccgcctcctcttgggttgtttggagcttcacatgtaatt |
| | | cacatgtaacatgtaacttgatcggtcagtgttcagaatgacaagtaac |
| | | cccgcttaaacttggtagaaggatggcccttagacctgaatggggtgat |
| | | tttacttgggatttaacttcttcagcaaattaacagcaacgttggaaga |
| | | gatctgtggcgcctctgtgaagcacaccgtgactcaggccagtctttta |
| | | gtgcagcgtgtctgggagtgaagggttttgcccttgctggtcttggagt |
| | | ccacagtgtgaggggcactgcacatgcctgggcatctacctagtgtgct |
| | | atgttcagtgtctggggcttactgccccggggtcctttcctctgggtgt |
| | | tggggcacagggtgctatgggaggcccatttgcttccctctcggagctc |
| | | agtttttgcttcatgggtcaaaatgtgggctggccaagtggttacagga |
| | | acagggtttcggtaagctatgttgtcttttttttttttttttttttttt |
| | | ttttaatggtttgattttgtgctgtggtattttttttcccttagaataa |
| | | tttttaatggcaaaacaggccttacagcagttgcttttctttaccattt |
| | | atttctttaagaagctttaaaatatttattgaaaagtgccatatctaat |
| | | ttctttagctttcgcctcaggcagtgcaggcatctttacttttcatcct |
| | | cagaagaaacaaacgactaacaaatgtagcaaatttactgcaggaatag |
| | | ttaggtcatgatactacctgaacactaaaccccagcctctttgtttggt |
| | | tttagttcctctgggtggtttttcttttgtgtgctggcttgattcttgt |
| | | gagaagttttgacctggccaagggagggttgagccatggttctggtgtg |
| | | ggactttgcggtcaagacacagtacagacaggtcaggcctgcgtgcctt |
| | | ttctctgggtggcctccccgttaggcccaccgtacgctcagccactata |
| | | gtgtccctgtggggccttgccatcagattgtgtgtcaggagatggtacc |
| | | tttttggtgtggctggggaggagtgtggtccatgccagttctttgggct |
| | | tcaggccactcttcccctcatgctgtggtgtaaagtgcacccatcaggt |
| | | ggtatatctggttctgatggcaagaagaaggtgggggatctccttatag |
| | | ggcatgggtctaggagcacagatgggccttttgccccgggtaaatgctt |
| | | gtctgtttgctgtcatgtgttctttgaggagtgagccatctcgagccct |
| | | gctttgaatttactgggtcatagagcctctgcctgtgctcttttccata |
| | | atgacttcatgtgacatgcacttttggtgggctcagataattggtttct |
| | | ttttgtttttgacctcaggctctgtggcagactggggaaaatggggcct |
| | | ggcatcattttccctgtcaatgggaggggctgttccatgcagggtggga |
| | | ggggaccaagttagcagagagtagccaaggatccttgcttcttcctttc |
| | | tagtgtgctgtcatccaagcaggctcctggctgtagggatgggccttgg |
| | | ggaagaatcttctttgaaagcatctatgataactgagaagtcatcccta |
| | | gttggagaaatccagtaatgagcagaaggaggaagcaagtgaggacaga |
| | | ggccattgtattacagtgtcacgcagagggccctcaatgatggggcatt |
| | | ggggaaggctgtagacatagtcatcagaacatcctggcctggcataagc |
| | | tgggttttctcctgggaccattggtcctcagcaggagttctttgcatga |
| | | gttgctcaggggcaagggctgcaagtgggctgtgcttaggagaaagtga |
| | | cacctggcagtgagggaagatggtgagcattattagcctttgttgtcca |
| | | gcatggccttcttgtcctgtctgctctggagaggagcctgtgggaccag |
| | | tcctgcctggggagggcatacccacacgtgccagctgattctgactctg |
| | | aatacatcatgtccggacttgggggtgtttctgcagaaaaaggaggttg |
| | | tttttcagccttgaacatcttcaggaggatagagactcttgctcacata |
| | | ttcttagcaaagggaagggtctctcatctccaggccacagagatagttc |
| | | ttccattgccctaagaggctaggctaaccctcttgacataacttagaca |
| | | gcaaagcacttcatcctgtagttgggctctgtcacctttctcttcagtt |
| | | ggccacattctcgtttcctccatcctgctatgctttgtgtgctcgggct |
| | | gtgtgtggggtttttccctggtggaaggaagcccagctgtgtattgaat |
| | | gtccttcatgtgttgtgtgtggctcagaaagcctgtcacttggcccctg |
| | | tgctctgagccgtgagggtggggaggtggctgttccattaaagtgggag |
| | | tattggatggccctcttgaaactagaattttgccttttttagtatgcag |
| | | tataaagtttccagcatctattggtaacacaaagatttgctggttttta |
| | | aaataatacagtaagcataagtatgtaagtttttagaattggtactaga |
| | | agttggacagctagttattctcgagaactttatttcactagaaaaatat |
| | | actaattggaaagcagtttccaggagttaactcagtttaattttcagtc |
| | | tcagttattttagcctgttgagtttttgatggcacacctttggagagat |
| | | ggccacgcctgattcccatttcaggggcatcagaccatacctttttaag |
| | | aagctccgtgaatctagtcatctacccttcatcctgggcgaacagccaa |
| | | aaagagaaggggacaaggtgtctttttctccttctcactggggtgacat |
| | | gaattcttttagttaatggctgtttgcaaattctaaactaatgaaatac |
| | | ttagcagctaacatgttcaatctagtaatgatgagtttaaatctcaatt |
| | | gacagtaatgttttagataaacaggcccagtaattcagttgatgaactg |
| | | tatatcttctcagtctagatttgtaaatgtttaatgaattcagggttat |
| | | aagcatagttctttaagtaagattccagatagttgatttgcaaccagca |
| | | gtctacctatgaatgtatcccaaacctttagaagattggaaaagatttt |
| | | tgaaataatgatttagttttgtaggaaaaacacccccttgaaaattaat |
| | | tcggttgacccagtaacattttttaaaacaattggtggctccaaaaggc |
| | | ctgccaacaaagaaaagtccaaattatctagtgggacattttgaatgtt |
| | | ttatgtttattttgggtccactgtaaactttggttcaaaaaagaatttg |
| | | aatttaaagaatttaccattatttaaattattaccaagtttttacattt |
| | | tcatgatggtattttccaggtatgaatgaaacatgactttttgattgtg |
| | | gtacttcctgtatcccctgtagtgccaaaaccagtgatactttatttgc |
| | | tcctatggcagctcatagaggtaaccgaagtgatttttcctcagtaatt |
| | | gaaacacatattctctaaatgccaatgtgtggtgatgggccctgcactg |
| | | ccttcatttctctagggcagtgtctttggattgtctagggcctaggtaa |
| | | ttctgagaactactgtaaaccaaccacagggcactaaagcaatgtacac |
| | | accactctttgtgtgtatggaaggggttatataaacctgggctatgctg |
| | | gacatctacagaagagtattacattcacttgcaaagtttacatttttga |
| | | gctcacagttatgaaaaatatgacccacaagtttttcaggcaggtgagg |
| | | atgggtcttcttgcaaatgcatgagttctgtcttgagtcctgggaactt |
| | | ctctgttggttgagtgtgggctcattccctgactctcctaatcatgttt |
| | | gcgtcagaatgttagcattgtaaataaaagaataggttgtataatagat |
| | | acacaacacttgaaactttactttaaaaaaatcgatagttctacatata |
| | | tatttagttatatcacttgacagatttcttctacacagtgtggagattg |
| | | ttttataccacagattatttttataaagttagtgaatttgaatgatttt |
| | | gtaatcagagctaatgagctttacctttcaagagaaacgtacactggag |
| | | catgagtggtgtggaacttttacttagtgtttatatggattcttgtgat |
| | | acactggcagactggagtcaatttgcgggtcttttttggccaaaactcc |
| | | acttgtggttgtgtaggacagtgatattcagctcagcttcttgtggatt |
| | | gggaggagagagggcctgcaatgtgttttacattggtgcttcctcctga |
| | | gatttctgttgaacaaagggttctgaggtcaaaaattagtttgtaagcc |
| | | tttgccataggacatagtcatgtgagagtgtttgggggaacagaaattg |
| | | tataggggtgcctattggggtgggatgggactcgaataagattcaggta |
| | | caaaaactttgaaatgagaatctggtggtttgagtaatccaccagactg |
| | | aattatctaagatcacattatccaggttggggggcagaattacccagtt |
| | | aagtaattgttcagaaaagtggggagggtggcatgtggatgcagtgatc |
| | | caattaaatggagagctgccaggcacattttgtcctctctggtcagtga |
| | | gaatggttgggttggctcgctgcttcaatctgtggaatcagccaggagc |
| | | ccagtgaggaagctcagaaccccagtaacagcagagcatctttcagata |
| | | gctccagagttttcctgcttttctgaggaagctcagcatcactgccaca |
| | | atacggaaagtggtcttcattttagcctatttatttttaggcagagagt |
| | | ggatggttatttgtgtgggacttttggtggcgatatataatgaataatt |
| | | aagttaatttctggtatgcataatggccagtcctgaggcccagctgaag |
| | | acctgtcccccagaccctgcccgctggcttcaggctgctgcttctagac |
| | | agaggtgcactggacgggatagttttatcaagagaatccctaatgtgtc |
| | | attttaaaccagctgtgctttttattcattctggttgagcgtataggtt |
| | | tacactttaccctttttatacttggaataaatttagttccagcagatct |
| | | agtagcactccagaaaccaaccccatctgttccccataaaaagaacatt |
| | | ttctctgctctccagccacgtgtcttggaatgtaattctgttgtgcctt |
| | | tgtttttatcactctcttcgccccaaaagcaactgctgtaagctttttt |
| | | ctacttgtcttttctagtccccaacctctacctttttcctttttcccag |
| | | ccctaatttctggatgcacttctgtgatccaggtattttaagaaccagt |
| | | tacctcagacctcatgttgaacagtgtcgccatctgggtcctcttgata |
| | | ctgcagacttttaacgtacacatgcaggaaccctgctgagcgtgggcac |
| | | ttgttttaaagcaaaactcttcccaaggactgaagaaagggcttctggc |
| | | aagctcgtcatggcattgtggtgggatgggtctagagtgtcatctgaat |
| | | ggtgcttcctgtgttcctctttgaattctgccattttcagtattcttgt |
| | | gtgtctgaataggcaaagcgatttaattggctggtcttgcacgcaaatt |
| | | agttccaaagataagctctttgtaacacatttccagtcgctaatgctca |
| | | aatgtagaacattcctttaaatggcaggataaaaaacccactatccacc |
| | | atagtgcattttgggaagatgtctgtagcatatgttgctgtgaaattag |
| | | gccttgtgggatatggctgtttgtcattttgatgtattttaaataaata |
| | | tatatattttttaaagagccttttttaccagttcaaaaagtttaattaa |
| | | ccagcagtcaccgcatctgaatttttgtctctggggcatagatggcaga |
| | | ccaagattaaaagtggtaactcagctatacgagcatgggctaccttcct |
| | | gggctctcctgcagtcctgtagacctgctgttccgcagaccatgggaca |
| | | caaggtcagtgtgttcccagtgagggtcccaagtcagtcatcttaagtg |
| | | tttgttctctgccccattcagtggactgttgacttcagtccctgcaagt |
| | | gctttagcccgagtggggttttctcagagcactgccacgagttaagtgt |
| | | gtgtttagccaaataatttctccgtaagggaaaaatgcagtcacccaaa |
| | | ttttaccaacaatgacagagatgagagtagaaaagattaggcaacatct |
| | | gagttttaacttgaaaagtgtccaagtcatcatgaaaggccgactggga |
| | | gcaagtgattattagagattcttcaggagacctcatctgaaaatgttaa |
| | | gactgccagtgagggaaggaattgttaaaatgccagcggcttttttttc |
| | | ctctttttttctgtaattctgtaaaaatgcagagaaagttgagtggtac |
| | | ttcagaattgagggagagggttaccgcagagtagaaatatatttctaga |
| | | tttcagttccacaccacaaatccacaacaatgccatttttcaactgtac |
| | | aaaaatctgcttatgaactggacatgatcttaatggtagtgtcaaaggc |
| | | caagtttttcacctgttaatatttttccacatttgtccttgaatctgaa |
| | | taactttatacagtactgtaaatttaacttacatcgagtttgttgtcaa |
| | | ttcttatgaaaagagctttctgcatgtaacacatacggttaaagaacac |
| | | agcaaaggacaaaatttgcaggaacagttttggaaccaacagaaaatgt |
| | | caccttttatttgccatcttatatatatctatcagttttaccagctact |
| | | tctaaatttgtacattatttgtaagggaaagaaggaaaaccctaagact |
| | | tgtctaacttagtggagaatgtgtgtgttgggcttaggatggatagcta |
| | | agtcttattgagctgtgttacctaacttgtatataaaaattgtaattaa |
| | | aagtttgggttcacctgtttctcacagtttaaaatgatgagtaattgca |
| | | aactctggaaatgtgactagtatatgatttaaggctgtagaagcaagga |
| | | agctctttcaagtgctaaaactaaagacttctagtttttggctcaaata |
| | | agtactgtttgtataccaggatatgtgagatgtaaatgtagtaggtcac |
| | | ttttcacccttgtagctataaaataaaaattttgtagaacagaaatagc |
| | | ttgtactactgaattaacaaaagttatactaaagtatcatgtttaaaaa |
| | | aaatatatatatatatacagagttaagcttgttgctgttaccctgtctg |
| | | gatttgaaaagtgtgctgatttatatatatatattacacacacacacac |
| | | acacacacacacacacacacacacacacacacacacacacacacacaca |
| | | cacacacacatacacctaaaatggcctaaagcagacatccatgtaatta |
| | | cagttgcaaaatgaaaacattttggaaagaacattgtatcatagttcat |
| | | tcatttgcagtggatctttgttcctttttactgtggtaattttagaaat |
| | | gagtgtcaagtttgaaattagatctgctaagttggggttttgctgcttg |
| | | aactctgcactgggtcctcaaataaaccgatgtgaatgtagttttttcc |
| | | ccctgtgtgaagaagcagttacaccccaacaataggaggaaaaatctag |
| | | aactatttcaagttttatctttttgtatatgaaaataaaataataataa |
| | | aacaa |

RNAscope® Assay
   RNAscope® 2.0 HD Red Reagent Kit (Cat. No 310036)

Quality Control (QC)

QC Purpose: Sample QC was performed first to assess the overall quality and integrity of RNA in the tissue samples and fixation conditions.

QC Process: RNA polymerase II polypeptide A (PolR2A) and cyclophilin B (PPIB) are low-copy (10-20 copies per cell) housekeeping genes that serve as a rigorous test of tissue RNA integrity. Bacterial dapB is a non-specific probe that will generate no background signal on properly fixed tissue. This was used to assess fixation and verify technical accuracy of the method.

Sample QC PASS:
≧2 PolR2A/PPIB score—relatively uniform PolR2A/PPIB signal throughout the samples
AND
<1 dapB score—no non-specific signal from dapB verifies that all detected signal with a target-specific probe will accurately reflect target RNA expression.

Technical QC: To verify that the RNAscope method was performed with technical accuracy, reference slides were tested with PolR2A/PPIB and dapB/dapB in parallel with tissue sample QC. Reference slides consisting of FFPE cell pellets showing 3+ PolR2A/PPIB and 0 dapB/dapB established that the assay performance passed QC.

Sample Scoring Criteria:

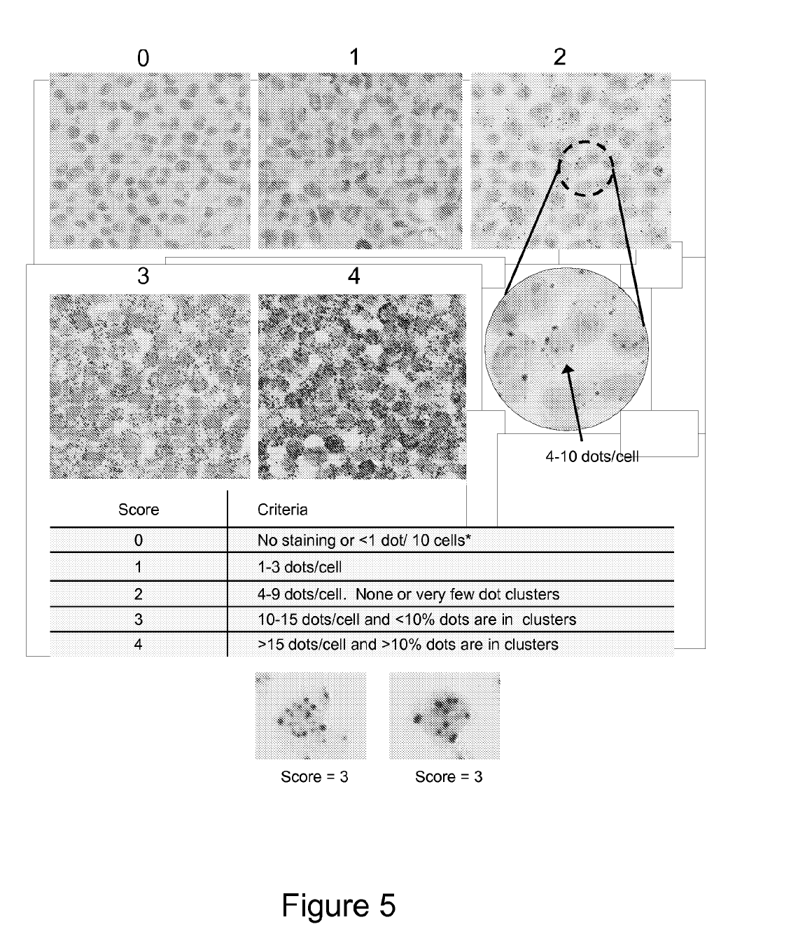

Samples were scored on a scale of 0 to 4. The table below shows the criteria for assigning each score to a given sample. See FIG. 5.

| Score | Criteria |
|---|---|
| 0 | No staining or <1 dot/10 cells* |
| 1 | 1-3 dots/cell |
| 2 | 4-9 dots/cell. None or very few dot clusters |
| 3 | 10-15 dots/cell and <10% dots are in clusters |
| 4 | >15 dots/cell and >10% dots are in clusters |

If <5% of cells score 1 and >95% of cells score 0, a score of 0 was given. If 5-30% of cells score 1 and >70% of cells score 0, a score of 0.5 was given. Scoring was performed at 20× magnification.

Note: When interpreting RNAscope staining scoring the number of dots per cell can be performed or the signal intensity can be used. Dots correlate to the number of individual RNA molecules, whereas dot intensity reflects the number of probe pairs bound to each molecule.

Phase I:

One (1) FFPE TMA sample was evaluated by RNAscope ISH.

Hs-PolR2A/Hs-PPIB was used as a positive control marker for sample QC and to evaluate RNA quality in both cell pellets and tissue samples (see probe information above). Bacterial gene dapB was used as a negative control. Semi-quantitative scores (scale of 0-4) were obtained for all samples to assess sample quality and to determine QC pass/fail.

Positive control scores ranged from 0-4, indicating a range in RNA quality. See table below. A significant number of cores (4) showed little to no positive control staining and failed QC. DapB scores were 0, indicating no or negligible non-specific background. See the Sample Scoring Criteria, above.

| | | | POLR2A/ | dapB/ | |
|---|---|---|---|---|---|
| | | | PPIB | dapB | |
| Sample | | | score | score | QC |
| Name | Tissue type | Sample ID | (pos ctl) | (neg ctl) | Pass/Fail |
| NMLT | Xenograft | Core 1A | 0/0 | 0/0 | Fail |
| | TMA | | | | |
| TOV-21G | Xenograft | Core 1B | 2/1 | 0/0 | Pass |
| | TMA | | | | (marginal) |
| ST013 | Xenograft | Core 1C | 2/3 | 0/0 | Pass |
| | TMA | | | | |
| ST860 | Xenograft | Core 1D | 1/2 | 0/0 | Fail |
| | TMA | | | | |
| ST1473 | Xenograft | Core 1E | 0/0 | 0/0 | Fail |
| | TMA | | | | |
| ST182 | Xenograft | Core 2A | 2/4 | 0/0 | Pass |
| | TMA | | | | |
| ST1469 | Xenograft | Core 2B | 2/2 | 0/0 | Pass |
| | TMA | | | | |
| ST844 | Xenograft | Core 2C | 0/0 | 0/0 | Fail |
| | TMA | | | | |

Standard pretreatment assay conditions were determined to be optimal for the samples in this study set.

Conclusion: 4 out of 8 cores (50%) scored as QC Pass for RNAscope analysis (see Scoring Table above).

Phase II:

RNAscope 2-plex Assay was performed to evaluate Hs-INHBA and Hs-ACVR2B expression in the one TMA sample. Both genes were detected at low to moderate levels in the cores that passed QC. See table below. RNAscope ISH staining for Hs-INHBA and Hs-ACVR2B expression were scored semi-quantitatively in all cores (see scoring table below).

| Sample | | | | INHBA | ACVR2B |
|---|---|---|---|---|---|
| Name | Tissue type | Sample ID | QC Pass/Fail | (green) | (red) |
| NMLT | Xenograft | Core 1A | Fail | 1 | 0 |
| | TMA | | | | |
| TOV-21G | Xenograft | Core 1B | Pass | 0 | 0 |
| | TMA | | (marginal) | | |
| ST013 | Xenograft | Core 1C | Pass | 1 | 1 |
| | TMA | | | | |
| ST860 | Xenograft | Core 1D | Fail | 0 | 0 |
| | TMA | | | | |
| ST1473 | Xenograft | Core 1E | Fail | 0 | 0 |
| | TMA | | | | |
| ST182 | Xenograft | Core 2A | Pass | 2 | 1 |
| | TMA | | | | |
| ST1469 | Xenograft | Core 2B | Pass | 2 | 2 |
| | TMA | | | | |
| ST844 | Xenograft | Core 2C | Fail | 0 | 0 |
| | TMA | | | | |

Conclusion: Hs-INHBA and Hs-ACVR2B were evaluated using the RNAscope 2plex chromogenic assay in one TMA sample. Both genes were able to be co-detected in cores that passed QC.

### Example 7

Chromogenic 2-plex RNASCOPE® ISH Assay Development and Validation of Human INHBA and ACVR2B Target Genes in Multiple Tumor Samples.

Objective:

Develop and demonstrate validation of Chromogenic 2-plex RNAscope In Situ Hybridization (ISH) assay for detection of Human Activin A (INHBA) and Activin A receptor type IIB (ACVR2B) in multiple distinct tumor types.

Project Scope:

Phase I: Sample Validation and Optimization
Evaluate sample quality using positive and negative reference controls.

Phase II: Marker Evaluation and Data Analysis

Perform RNAscope assay on qualified samples using target probe, collect images, score, and analyze data.

Protocols, Materials, and Methods:

These were performed as described in Example 6, with the exception of sample source(s). Samples analyzed in this example were 7 TMA samples with 8-9 cores each. Tumor types included ovary, endometrial, pancreas, bile duct, lung, gastric, head/neck, breast, colorectal, melanoma, and testicular. The table below provides further detail on tumor types and sub-types.

Sample Scoring Criteria:

Scoring was performed as described in Example 6.

Phase I:

Seven (7) FFPE multi-tissue TMA samples were prepared by START Research Center and evaluated at ACD Bio by RNAscope ISH.

Hs-PolR2A/Hs-PPIB was used as a positive control marker for sample QC and to evaluate RNA quality in all seven TMAs. Bacterial gene dapB was used as a negative control.

Semi-quantitative scores (scale of 0-4) were obtained for all samples to assess sample quality and to determine QC pass/fail. Positive control scores were mostly 3, indicating good RNA quality in most cores. A number of cores scored lower than 1 due to low positive control staining and failed QC, suggesting compromised RNA quality in these cores. Some cores on Array 6 (breast tissue) had background staining of dapB; those that scored 1 also failed QC. See table below.

| | | | | PolR2A/ | dapB/ | | |
|---|---|---|---|---|---|---|---|
| | | | | PPIB | dapB | QC | |
| | | | | score | score | Pass/ | |
| Array | Model | Primary | Sub-Type | (pos ctl) | (neg ctl) | Fail | Notes |
| 1 | ST1162 | Ovary | Papillary serous carcinoma | 3 | 0 | P | |
| 1 | ST255B | Ovary | Papillary serous carcinoma | 3 | 0 | P | |
| 1 | ST097 | Ovary | Papillary serous carcinoma | 2 | 0 | P | Low |
| | | | | | | | PolR2A |
| | | | | | | | staining |
| 1 | ST022 | Ovary | Papillary serous carcinoma | 3 | 0 | P | |
| 1 | ST270 | Ovary | Papillary serous carcinoma | 2 | 0 | P | |
| 1 | ST1473 | Endometrial | Clear Cell Carcinoma | 2 | 0 | P | Partially |
| | | | | | | | detached |
| 1 | ST182 | Ovary | Fallopian tube; Serous | 3 | 0 | P | |
| | | | Papillary Carcinoma | | | | |
| 1 | ST182B | Ovary | Fallopian tube; Serous | 3 | 0 | P | |
| | | | Papillary Carcinoma | | | | |
| 2 | ST1584 | Pancreas | Adenocarcinoma | 3 | 0 | P | |
| 2 | ST1185 | Pancreas | Adenocarcinoma | 3 | 0 | P | |
| 2 | ST1317 | Pancreas | Adenocarcinoma | 3 | 0 | P | |
| 2 | ST828 | Pancreas | Adenocarcinoma | 3 | 0 | P | |
| 2 | ST1587 | Pancreas | Adenocarcinoma | 3 | 0 | P | Partially |
| | | | | | | | detached |
| 2 | ST1933 | Pancreas | Adenocarcinoma | 2 | 0 | P | |
| 2 | ST712 | Pancreas | Mixed ductal endocrine | 1 | 0 | F | Low pos |
| | | | carcinoma | | | | staining |
| 2 | ST174 | Bile Duct | Cholangiocarcinoma | 1 | 0 | F | Low pos |
| | | | | | | | staining |
| 3 | ST1271 | Lung | NSCLC-Adenocarcinoma | — | — | — | Missing |
| | | | | | | | core |
| 3 | ST209 | Lung | NSCLC-Adenocarcinoma | — | — | — | Missing |
| | | | | | | | core |
| 3 | ST1749 | Lung | NSCLC-Adenocarcinoma | 3 | 0 | P | |
| 3 | ST1748 | Lung | NSCLC-Adenocarcinoma | 3 | 0 | P | |
| 3 | ST751 | Lung | NSC-NEC | — | — | — | Missing |
| | | | | | | | core |
| 3 | ST115 | Lung | NSC-SCC | — | — | — | Missing |
| | | | | | | | core |
| 3 | ST1328 | Lung | NSC-SCC | 2 | 0 | P | |
| 3 | ST137 | Lung | SCLC | 0 | 0 | F | Low pos |
| | | | | | | | staining |
| 3 | ST171 | Lung | SCLC | — | — | — | Missing |
| | | | | | | | core |
| 4 | ST1386 | Endometrial | AC-Endometrioid | 3 | 0 | P | |
| 4 | ST259 | Endometrial | AC-Endometrioid | 3 | 0 | P | |
| 4 | ST993 | Endometrial | AC-Endometrioid | 3 | 0 | P | |
| 4 | ST1603 | Endometrial | AC-Endometrioid | 3 | 0 | P | Small |
| | | | | | | | cellular |
| | | | | | | | region |
| 4 | ST562 | Gastric | Adenocarcinoma | 3 | 0 | P | |
| 4 | ST167 | Gastric | GEJ Adenocarcinom | 1 | 0 | F | Low pos |
| | | | | | | | staining |
| 4 | ST183 | Gastric | Adenocarcinoma | 1 | 0 | F | Low pos |
| | | | | | | | staining |
| 4 | ST602 | Gastric | GEJ Adenocarcinom | 3 | 0 | P | |
| 4 | ST983 | Gastric | Duodenal Adenocarcinoma | 3 | 0 | P | |
| 5 | ST073 | Head/Neck | SCC Laryngeal | 2 | 0 | P | |
| 5 | ST1095 | Head/Neck | ACC Esophageal | 3 | 0 | P | |
| 5 | ST744 | Head/Neck | SCC Tongue | 2 | 0 | P | |
| 5 | ST1145 | Head/Neck | SCC Oropharynx | 3 | 0 | P | |
| 5 | ST1379B | Head/Neck | SCC Hypopharynx | 3 | 0 | P | |
| 5 | ST1725 | Head/Neck | SCC Oropharynx | 3 | 0 | P | |
| 5 | ST1502B | Head/Neck | ACC | 4 | 0 | P | |
| 5 | ST486 | Head/Neck | ACC | 3 | 0 | P | |
| 6 | ST2057 | Breast | Infiltrating Ductal | 2 | 1 | F | High neg |
| | | | Carcinom | | | | staining |
| 6 | ST575 | Breast | Infiltrating Ductal | 3 | 0 | P | |
| | | | Carcinom | | | | |
| 6 | ST1834 | Breast | Infiltrating Ductal | 3 | 1 | F | High neg |
| | | | Carcinom | | | | staining |
| 6 | ST616 | Breast | Infiltrating Ductal | 3 | 0 | P | |
| | | | Carcinom | | | | |
| 6 | ST1339 | Breast | Infiltrating Ductal | 3 | 0.5 | P | |
| | | | Carcinom | | | | |
| 6 | ST225 | Breast | Infiltrating Ductal | 1 | 0 | F | Low pos |
| | | | Carcinom | | | | staining |
| 6 | ST518 | Breast | Lobular Carcinoma | 2 | 0 | P | |
| 6 | ST1056 | Breast | Invasic Ductal Carcinoma | 3 | 1 | F | High neg |
| | | | | | | | staining |
| 7 | ST534 | Colorectal | Adenocarcinoma | 3 | 0 | P | |
| 7 | ST1406 | Colorectal | AC-Sigmoid | 2 | 0 | P | |
| 7 | ST512 | Colorectal | Adenocarcinoma | 3 | 0 | P | |
| 7 | ST1199 | Colorectal | Adenocarcinoma | 2 | 0 | P | |
| 7 | ST1310 | Colorectal | AC-Rectosigmoid | 2 | 0 | P | Partially |
| | | | | | | | detached |
| 7 | ST1281 | Colorectal | AC-Sigmoid | 4 | 0 | P | Partially |
| | | | | | | | detached |
| 7 | ST094 | Colorectal | AC-Rectal | 1 | 0 | F | Low pos |
| | | | | | | | staining |
| 7 | ST1411 | Melanoma | Nodular Melanoma | 3 | 0 | P | |
| 7 | ST1908 | Testicular | Mixed Germ Cell tumor | 4 | 1 | F | High neg |
| | | | | | | | staining |

Standard pretreatment assay conditions were determined to be optimal for the samples in this study set.

Conclusion: 43 out of 59 cores (73%) scored as QC Pass for RNAscope analysis. 7 out of 59 cores (12%) failed QC due to low positive control staining. 4 out of 59 cores (7%) failed due to high background staining 5 out of 59 cores (8%) were missing from the original TMA block on Array 3. See QC Scoring Table shown above.

Phase II:

RNAscope 2-plex Assay was performed to evaluate Hs-ACVR2B and Hs-INHBA expression in all seven TMA samples. Both genes were detected at low to moderate levels (scores 0-3) in the cores that passed QC. See table below. RNAscope ISH staining for Hs-ACVR2B and Hs-INHBA expression were scored semi-quantitatively in all cores.

| | | | | QC | ACVR | | |
|---|---|---|---|---|---|---|---|
| | | | | Pass/ | 2B | INHBA | |
| Array | Model | Primary | Sub-Type | Fail | score | score | Notes |
| 1 | ST1162 | Ovary | Papillary serous | P | 1 | 1 | |
| | | | carcinoma | | | | |
| 1 | ST255B | Ovary | Papillary serous | P | 1 | 3 | |
| | | | carcinoma | | | | |
| 1 | ST097 | Ovary | Papillary serous | P | 0 | 1 | |
| | | | carcinoma | | | | |
| 1 | ST022 | Ovary | Papillary serous | P | 1 | 2 | |
| | | | carcinoma | | | | |
| 1 | ST270 | Ovary | Papillary serous | P | 1 | 1 | |
| | | | carcinoma | | | | |
| 1 | ST1473 | Endometrial | Clear Cell Carcinoma | P | 0 | 0 | Partially |
| | | | | | | | detached |
| 1 | ST182 | Ovary | Fallopian tube; Serous | P | 1 | 2 | |
| | | | Papillary Carcinoma | | | | |
| 1 | ST182B | Ovary | Fallopian tube; Serous | P | 1 | 2 | |
| | | | Papillary Carcinoma | | | | |
| 2 | ST1584 | Pancreas | Adenocarcinoma | P | 1 | 1 | |
| 2 | ST1185 | Pancreas | Adenocarcinoma | P | 0 | 3 | |
| 2 | ST1317 | Pancreas | Adenocarcinoma | P | 0 | 3 | |
| 2 | ST828 | Pancreas | Adenocarcinoma | P | 1 | 2 | |
| 2 | ST1587 | Pancreas | Adenocarcinoma | P | 1 | 3 | Partially |
| | | | | | | | detached |
| 2 | ST1933 | Pancreas | Adenocarcinoma | P | 0 | 32 | |
| 2 | ST712 | Pancreas | Mixed ductal endocrine | F | 0 | 1 | |
| | | | carcinoma | | | | |
| 2 | ST174 | Bile Duct | Cholangiocarcinoma | F | 1 | 1 | |
| 3 | ST1271 | Lung | NSCLC-Adenocarcinoma | — | — | — | Missing core |
| 3 | ST209 | Lung | NSCLC-Adenocarcinoma | — | — | — | Missing core |
| 3 | ST1749 | Lung | NSCLC-Adenocarcinoma | P | 1 | 2 | |
| 3 | ST1748 | Lung | NSCLC-Adenocarcinoma | P | 1 | 1 | |
| 3 | ST751 | Lung | NSC-NEC | — | — | — | Missing core |
| 3 | ST115 | Lung | NSC-SCC | — | — | — | Missing core |
| 3 | ST1328 | Lung | NSC-SCC | P | 2 | 1 | |
| 3 | ST137 | Lung | SCLC | F | 1 | 1 | |
| 3 | ST171 | Lung | SCLC | — | — | — | Missing core |
| 4 | ST1386 | Endometrial | AC-Endometrioid | P | 1 | 0 | |
| 4 | ST259 | Endometrial | AC-Endometrioid | P | 0 | 0 | |
| 4 | ST993 | Endometrial | AC-Endometrioid | P | 1 | 1 | |
| 4 | ST1603 | Endometrial | AC-Endometrioid | P | 1 | 1 | Small |
| | | | | | | | cellular |
| | | | | | | | region |
| 4 | ST562 | Gastric | Adenocarcinoma | P | 0 | 2 | |
| 4 | ST167 | Gastric | GEJ Adenocarcinom | F | 0 | 0 | |
| 4 | ST183 | Gastric | Adenocarcinoma | F | 0 | 0 | |
| 4 | ST602 | Gastric | GEJ Adenocarcinom | P | 1 | 3 | |
| 4 | ST983 | Gastric | Duodenal | P | 1 | 1 | |
| | | | Adenocarcinoma | | | | |
| 5 | ST073 | Head/Neck | SCC Laryngeal | P | 1 | 3 | |
| 5 | ST1095 | Head/Neck | ACC Esophageal | P | 2 | 3 | |
| 5 | ST744 | Head/Neck | SCC Tongue | P | 1 | 3 | |
| 5 | ST1145 | Head/Neck | SCC Oropharynx | P | 1 | 3 | |
| 5 | ST1379B | Head/Neck | SCC Hypopharynx | P | 1 | 3 | |
| 5 | ST1725 | Head/Neck | SCC Oropharynx | P | 1 | 2 | |
| 5 | ST1502B | Head/Neck | ACC | P | 2 | 2 | |
| 5 | ST486 | Head/Neck | ACC | P | 2 | 3 | |
| 6 | ST2057 | Breast | Infiltrating Ductal | F | 1 | 2 | |
| | | | Carcinom | | | | |
| 6 | ST575 | Breast | Infiltrating Ductal | P | 1 | 2 | |
| | | | Carcinom | | | | |
| 6 | ST1834 | Breast | Infiltrating Ductal | F | 0 | 2 | |
| | | | Carcinom | | | | |
| 6 | ST616 | Breast | Infiltrating Ductal | P | 0 | 2 | |
| | | | Carcinom | | | | |
| 6 | ST1339 | Breast | Infiltrating Ductal | P | 1 | 2 | |
| | | | Carcinom | | | | |
| 6 | ST225 | Breast | Infiltrating Ductal | F | 0 | 0 | |
| | | | Carcinom | | | | |
| 6 | ST518 | Breast | Lobular Carcinoma | P | 0 | 2 | |
| 6 | ST1056 | Breast | Invasic Ductal Carcinoma | F | 1 | 2 | |
| 7 | ST534 | Colorectal | Adenocarcinoma | P | 0 | 0 | |
| 7 | ST1406 | Colorectal | AC-Sigmoid | P | 0 | 0 | |
| 7 | ST512 | Colorectal | Adenocarcinoma | P | 0 | 1 | |
| 7 | ST1199 | Colorectal | Adenocarcinoma | P | 0 | 0 | |
| 7 | ST1310 | Colorectal | AC-Rectosigmoid | P | 1 | 1 | Partially |
| | | | | | | | detached |
| 7 | ST1281 | Colorectal | AC-Sigmoid | P | 2 | 1 | Partially |
| | | | | | | | detached |
| 7 | ST094 | Colorectal | AC-Rectal | F | 0 | 1 | |
| 7 | ST1411 | Melanoma | Nodular Melanoma | P | 1 | 2 | |
| 7 | ST1908 | Testicular | Mixed Germ Cell tumor | F | 3 | 2 | |

Scores are presented for the entire tissue. Note that cellular heterogeneity in expression was observed, with a subset of cells showing higher expression.

Conclusion: Using the same probe set previously developed in Example 6, Hs-ACVR2B and Hs-INHBA were evaluated using the RNAscope 2plex chromogenic assay in seven new multi-tissue TMA samples. The majority of cores passed QC, and both genes were detected at low to moderate levels in cores that passed QC. Overall, INHBA was expressed at higher levels than ACVR2B in most tissues.

The table below identifies sequences listed in the sequence listing.

| SEQ | |
|---|---|
| ID NO | Description |
| 1 | ActRIIB extracellular domain, polynucleotide |
| 2 | ActRIIB extracellular domain, polypeptide |
| 3 | svActRIIB (E28W, S44T) polynucleotide with signal sequence |
| 4 | svActRIIB (E28W, S44T) polypeptide with signal sequence |
| 5 | svActRIIB (E28W, S44T) polynucleotide without signal |
| | sequence |
| 6 | svActRIIB (E28W, S44T) polypeptide without signal sequence |
| 7 | svActRIIB-Fc (E28W, S44T) polynucleotide with signal |
| | sequence |
| 8 | svActRIIB-Fc (E28W, S44T) polypeptide with signal sequence |
| 9 | svActRIIB-Fc (E28W, S44T) polynucleotide without signal |
| | sequence |
| 10 | svActRIIB-Fc (E28W, S44T) polypeptide without signal |
| | sequence |
| 11 | svActRIIB (E28Y, S44T) polynucleotide with signal sequence |
| 12 | svActRIIB (E28Y, S44T) polypeptide with signal sequence |
| 13 | svActRIIB (E28Y, S44T) polynucleotide without signal |
| | sequence |
| 14 | svActRIIB (E28Y, S44T) polypeptide without signal sequence |
| 15 | svActRIIB-Fc (E28Y, S44T) polynucleotide with signal |
| | sequence |
| 16 | svActRIIB-Fc (E28Y, S44T) polypeptide with signal sequence |
| 17 | svActRIIB-Fc (E28Y, S44T) polynucleotide without signal |
| | sequence |
| 18 | svActRIIB-Fc (E28Y, S44T) polypeptide without signal |
| | sequence |
| 19 | ActRIIB (E28W) polypeptide, without signal sequence |
| 20 | ActRIIB-Fc (E28W) polynucleotide, without signal sequence |
| 21 | ActRIIB-Fc (E28W) polypeptide, without signal sequence |
| 22 | IgG2Fc polypeptide sequence |
| 23 | IgG1Fc polypeptide sequence |
| 24 | IgG4 Fc polypeptide sequence |
| 25 | Linker amino acid sequence |
| 26 | Hinge linker #1 polynucleotide sequence |
| 27 | Hinge linker #1 peptide sequence |
| 28 | Hinge region IgG2 |
| 29 | Hinge region IgG1 |
| 30 | Hinge region IgG4 |
| 31 | Alternative signal sequence, polypeptide |
| 32 | Signal sequence, polypeptide |
| 33 | Wild type ActRIIB accession NP_001097 |
| 34 | Activin polypeptide sequence |
| 35 | Myostatin polypeptide sequence |
| 36 | GDF-11 polypeptide sequence |
| 37 | Hinge linker sequence #2 polynucleotide |
| 38 | Hinge linker sequence #2 peptide |
| 39 | Hinge linker sequence #3 polynucleotide |
| 40 | Hinge linker sequence #3 peptide |
| 41 | Hinge linker sequence #4 polynucleotide |
| 42 | Hinge linker sequence #4 peptide |
| 43 | Hinge linker sequence #5 polynucleotide |
| 44 | Hinge linker sequence #5 peptide |
| 45 | Hinge linker sequence #6 peptide |
| 46 | Hinge linker sequence #7 peptide |
| 47 | Modified IgG1 Fc polypeptide sequence |
| 48 | Hinge linker sequence #8 peptide |
| 49 | Hinge linker sequence #9 peptide |
| 50 | Hinge linker sequence #10 peptide |

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

All references, issued patents and patent applications cited within the body of the instant specification are hereby incorporated by reference in their entirety, for all purposes.

1. A method for treating a subject with an activin inhibitor, comprising:
performing an assay on a sample from the subject to generate a dataset comprising data representing the expression of at least two markers comprising inhibin beta A (INHBA) and activin A receptor type IIB (ACVR2B);
determining, based on the dataset, the likelihood that the subject will respond to treatment with the activin inhibitor, wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; and
administering the activin inhibitor to the subject if there is detectable expression of INHBA and ACVR2B within the sample.

2. The method of claim 1, wherein the assay is an in situ hybridization assay performed using a plurality of distinct probes, wherein the subject has cancer, wherein the sample comprises one or more cancer cells, wherein the cancer cells are ovary, endometrial, pancreas, bile duct, lung, gastric, head/neck, breast, colorectal, melanoma, or testicular cancer cells, and wherein the activin inhibitor is a polypeptide comprising the amino acid sequence set forth in SEQ ID NO:10.

3. The method of any one of claims 1-2, wherein the assay is a nucleotide-based assay, optionally wherein the nucleotide-based assay is an in situ hybridization assay performed using a plurality of distinct probes, optionally wherein the plurality of distinct probes hybridize to the nucleotides located at positions 364-1374 of the nucleotide sequence shown in SEQ ID NO:51 or the nucleotides located at positions 627-1503 of the nucleotide sequence shown in SEQ ID NO:52.

4. The method of any one of claims 1-3, wherein the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

5. The method of any one of claims 1-4, wherein the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

6. The method of any one of claims 1-5, wherein the subject is a human subject.

7. The method of any one of claims 1-6, wherein the subject has cancer.

8. The method of any one of claims 1-7, wherein the dataset further comprises data representing the location of the at least two markers relative to each other, optionally wherein co-localization of the markers indicates that the subject is more likely to be responsive to treatment with the activin inhibitor, optionally wherein the co-localization of the markers is in a single cell indicating autocrine signaling, and optionally wherein the co-localization of the markers is in neighboring cells indicating paracrine signaling.

9. A method for determining the likelihood that a subject will respond to treatment with an activin inhibitor, comprising:
performing an assay on a sample from the subject to generate a dataset comprising data representing the expression of at least two markers comprising INHBA and ACVR2B; and
determining, based on the dataset, the likelihood that the subject will respond to treatment with the activin inhibitor, wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment.

10. The method of claim 9, wherein the assay is an in situ hybridization assay performed using a plurality of distinct probes, wherein the subject has cancer, wherein the sample comprises RNA from one or more cancer cells, wherein the cancer cells are ovary, endometrial, pancreas, bile duct, lung, gastric, head/neck, breast, colorectal, melanoma, or testicular cancer cells, and wherein the activin inhibitor is a polypeptide comprising the amino acid sequence set forth in SEQ ID NO:10.

11. The method of any one of claims 9-10, wherein the assay is a nucleotide-based assay, optionally wherein the nucleotide-based assay is an in situ hybridization assay performed using a plurality of distinct probes, optionally wherein the plurality of distinct probes hybridize to the nucleotides located at positions 364-1374 of the nucleotide sequence shown in SEQ ID NO:51 or the nucleotides located at positions 627-1503 of the nucleotide sequence shown in SEQ ID NO:52.

12. The method of any one of claims 9-11, wherein the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

13. The method of any one of claims 9-12, wherein the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

14. The method of any one of claims 9-13, wherein the subject is a human subject.

15. The method of any one of claims 9-14, wherein the subject has cancer.

16. The method of any one of claims 9-15, further comprising administering the activin inhibitor to the subject.

17. The method of any one of claims 9-16, wherein the dataset further comprises data representing the location of the at least two markers relative to each other, optionally wherein co-localization of the markers indicates that the subject is more likely to be responsive to treatment with the activin inhibitor, optionally wherein the co-localization of the markers is in a single cell indicating autocrine signaling, and optionally wherein the co-localization of the markers is in neighboring cells indicating paracrine signaling.

18. A method for assaying the expression of INHBA and ACVR2B in a sample from a subject, comprising:
performing an in situ hybridization assay on the sample using a plurality of distinct probes that hybridize to the nucleotides located at positions 364-1374 of the nucleotide sequence shown in SEQ ID NO:51 or the nucleotides located at positions 627-1503 of the nucleotide sequence shown in SEQ ID NO:52; and
determining the expression of INHBA and ACVR2B based on the assay, wherein detectable expression of INHBA and ACVR2B within the sample indicates that the subject is more likely to be responsive to treatment with an activin inhibitor, and/or wherein the lack of detectable expression of at least one of INHBA and ACVR2B within the sample indicates that the subject is less likely to be responsive to treatment with the activin inhibitor.

19. The method of claim 18, wherein the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

20. The method of any one of claims 18-19, wherein the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

21. The method of any one of claims 18-20, wherein the subject is a human subject.

22. The method of any one of claims 18-21, wherein the subject has cancer.

23. The method of any one of claims 18-22, further comprising administering the activin inhibitor to the subject.

24. The method of any one of claims 18-23, wherein co-localization of the markers indicates that the subject is more likely to be responsive to treatment with the activin inhibitor, optionally wherein the co-localization of the markers is in a single cell indicating autocrine signaling, and optionally wherein the co-localization of the markers is in neighboring cells indicating paracrine signaling.

25. A method for determining the likelihood that a subject will respond to activin inhibitor therapy, comprising:
obtaining a dataset obtained from a sample from the subject, wherein the dataset comprises data representing the expression of at least two markers comprising INHBA and ACVR2B and, optionally, data representing the relative location of each of the markers within the sample; and
determining, based on the dataset, the likelihood that the subject will respond to activin inhibitor therapy,
wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; or
wherein higher expression of at least one of INHBA and ACVR2B relative to a control in combination with co-localization of INHBA and ACVR2B within the sample indicates that the subject is more likely to be responsive to activin inhibitor therapy than a second subject lacking at least one of higher expression of at least one of INHBA and ACVR2B relative to a control and co-localization of INHBA and ACVR2B within the sample.

26. The method of claim 25, wherein the method is implemented on a computer.

27. The method of claim 25, wherein obtaining the dataset obtained from the sample comprises obtaining the sample and processing the sample to experimentally determine the dataset; or wherein obtaining the dataset obtained from the sample comprises receiving the dataset from a third party that has processed the sample to experimentally determine the dataset.

28. The method of claim 25, wherein the sample comprises RNA from a cancer cell.

29. The method of claim 25, wherein the data are hybridization data.

30. The method of claim 25, wherein the dataset is obtained stored on a storage memory.

31. The method of any one of claims 25-30, wherein the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

32. The method of any one of claims 25-31, wherein the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

33. The method of any one of claims 25-32, wherein the subject is a human subject.

34. The method of any one of claims 25-33, wherein the subject has cancer.

35. The method of any one of claims 25-34, further comprising administering the activin inhibitor to the subject.

36. A method of treating a subject with an activin inhibitor, comprising:
obtaining a dataset obtained from a sample from the subject, wherein the dataset comprises data representing the expression of at least two markers comprising INHBA and ACVR2B and, optionally, data representing the relative location of each of the markers within the sample;
determining, based on the dataset, the likelihood that the subject will respond to activin inhibitor therapy,
wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; or
wherein higher expression of at least one of INHBA and ACVR2B relative to a control in combination with co-localization of INHBA and ACVR2B within the sample indicates that the subject is more likely to be responsive to activin inhibitor therapy than a second subject lacking at least one of higher expression of at least one of INHBA and ACVR2B relative to a control and co-localization of INHBA and ACVR2B within the sample; and
administering the activin inhibitor to the subject.

37. The method of claim 36, wherein obtaining the dataset obtained from the sample comprises obtaining the sample and processing the sample to experimentally determine the dataset; or wherein obtaining the dataset obtained from the sample comprises receiving the dataset from a third party that has processed the sample to experimentally determine the dataset.

38. The method of claim 36, wherein the sample comprises RNA from a cancer cell.

39. The method of claim 36, wherein the data are hybridization data.

40. The method of claim 36, wherein the dataset is obtained stored on a storage memory.

41. The method of any one of claims 36-40, wherein the sample comprises one or more cancer cells, RNA from one or more cancer cells, one or more fibroblasts, one or more stromal fibroblasts, stroma, and/or cancer-associated reactive stroma, optionally wherein the cancer cells are ovary, endometrial, pancreas, bile duct, lung, gastric, head/neck, breast, colorectal, melanoma, or testicular cancer cells.

42. The method of any one of claims 36-41, wherein the activin inhibitor comprises a polypeptide, wherein the polypeptide has an amino acid sequence with at least 95, 96, 97, 98, 99, or 100% sequence identity to the amino acid sequence set forth in SEQ ID NO:6, wherein the polypeptide has a W or a Y at the position corresponding to position 28 of the amino acid sequence set forth in SEQ ID NO:2, and a T at the position corresponding to position 44 of the amino acid sequence set forth in SEQ ID NO:2; optionally wherein the polypeptide further comprises a linker optionally having the amino acid sequence set forth in SEQ ID NO:27; optionally wherein the polypeptide further comprises a heterologous polypeptide optionally having the amino acid sequence set forth in SEQ ID NO:22; and/or optionally wherein the polypeptide comprises or consists of the amino acid sequence set forth in SEQ ID NO:10.

43. The method of any one of claims 36-42, wherein the subject is a human subject.

44. The method of any one of claims 36-43, wherein the subject has cancer.

45. A system for determining the likelihood that a subject will respond to activin inhibitor therapy, the system comprising:
a storage memory for storing a dataset obtained from a sample from the subject, wherein the dataset comprises data representing the expression of at least two markers comprising INHBA and ACVR2B and, optionally, data representing the relative location of each of the markers within the sample; and
a processor communicatively coupled to the storage memory for determining, based on the dataset, the likelihood that the subject will respond to activin inhibitor therapy,
wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; or
wherein higher expression of at least one of INHBA and ACVR2B relative to a control in combination with co-localization of INHBA and ACVR2B within the sample indicates that the subject is more likely to be responsive to activin inhibitor therapy than a second subject lacking at least one of higher expression of at least one of INHBA and ACVR2B relative to a control and co-localization of INHBA and ACVR2B within the sample.

46. The system of claim 45, wherein the dataset obtained from the sample comprises obtaining the sample and processing the sample to experimentally determine the dataset, or wherein the dataset obtained from the sample comprises receiving the dataset from a third party that has processed the sample to experimentally determine the dataset.

47. The system of claim 45, for implementation of any of the methods of claims 25-44.

48. A computer-readable storage medium storing computer-executable program code for scoring a sample obtained from a subject, the medium comprising:
a dataset obtained from a sample from the subject, wherein the dataset comprises data representing the expression of at least two markers comprising INHBA and ACVR2B and, optionally, data representing the relative location of each of the markers within the sample; and
computer-executable program code for determining, based on the dataset, the likelihood that the subject will respond to activin inhibitor therapy,
wherein detectable expression of INHBA and ACVR2B within the sample indicates that the subject is more likely to be responsive to treatment, and/or wherein the lack of detectable expression of at least one of INHBA and ACVR2B within the sample indicates that the subject is less likely to be responsive to treatment; or
wherein higher expression of at least one of INHBA and ACVR2B relative to a control in combination with co-localization of INHBA and ACVR2B within the sample indicates that the subject is more likely to be responsive to activin inhibitor therapy than a second subject lacking at least one of higher expression of at least one of INHBA and ACVR2B relative to a control and co-localization of INHBA and ACVR2B within the sample.

49. The medium of claim 48, for implementation of any of the methods of claims 25-44.


SEQUENCE LISTING (53 sequences)

SEQ ID NO: 1 - DNA, 402 bp, Homo sapiens
  CDS (1)..(402)
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccacc

SEQ ID NO: 2 - protein, 134 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 3 - DNA, 387 bp, Homo sapiens
  CDS (1)..(387)
atggagtttgggctgagctgggttttcctcgttgctcttttaagaggtgtccagtgtgagacacggtggtgcatctactacaacgccaactgggagctggagcgcaccaaccagaccggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgagggcaacttctgcaacgagcgcttcactcatttgccagaggctgggggcccggaagtcacgtacgagccacccccgacagcccccacc

SEQ ID NO: 4 - protein, 129 aa, Homo sapiens
MEFGLSWVFLVALLRGVQCETRWCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 5 - DNA, 330 bp, Homo sapiens
  CDS (1)..(330)
gagacacggtggtgcatctactacaacgccaactgggagctggagcgcaccaaccagaccggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgagggcaacttctgcaacgagcgcttcactcatttgccagaggctgggggcccggaagtcacgtacgagccacccccgacagcccccacc

SEQ ID NO: 6 - protein, 110 aa, Homo sapiens
ETRWCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 7 - DNA, 1071 bp, Homo sapiens
  CDS (1)..(1071)
atggagtttgggctgagctgggttttcctcgttgctcttttaagaggtgtccagtgtgagacacggtggtgcatctactacaacgccaactgggagctggagcgcaccaaccagaccggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgagggcaacttctgcaacgagcgcttcactcatttgccagaggctgggggcccggaagtcacgtacgagccacccccgacagcccccaccggagggggaggatctgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 357 aa, Homo sapiens
MEFGLSWVFLVALLRGVQCETRWCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPTGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - DNA, 1014 bp, Homo sapiens
  CDS (1)..(1014)
gagacacggtggtgcatctactacaacgccaactgggagctggagcgcaccaaccagaccggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgagggcaacttctgcaacgagcgcttcactcatttgccagaggctgggggcccggaagtcacgtacgagccacccccgacagcccccaccggagggggaggatctgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 338 aa, Homo sapiens
ETRWCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPTGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - DNA, 387 bp, Homo sapiens
  CDS (1)..(387)
atggagtttgggctgagctgggttttcctcgttgctcttttaagaggtgtccagtgtgagacacggtactgcatctactacaacgccaactgggagctggagcgcaccaaccagaccggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgagggcaacttctgcaacgagcgcttcactcatttgccagaggctgggggcccggaagtcacgtacgagccacccccgacagcccccacc

SEQ ID NO: 12 - protein, 129 aa, Homo sapiens
MEFGLSWVFLVALLRGVQCETRYCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 13 - DNA, 330 bp, Homo sapiens
  CDS (1)..(330)
gagacacggtactgcatctactacaacgccaactgggagctggagcgcaccaaccagaccggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgagggcaacttctgcaacgagcgcttcactcatttgccagaggctgggggcccggaagtcacgtacgagccacccccgacagcccccacc

SEQ ID NO: 14 - protein, 110 aa, Homo sapiens
ETRYCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 15 - DNA, 1071 bp, Homo sapiens
  CDS (1)..(1071)
atggagtttgggctgagctgggttttcctcgttgctcttttaagaggtgtccagtgtgagacacggtactgcatctactacaacgccaactgggagctggagcgcaccaaccagaccggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgagggcaacttctgcaacgagcgcttcactcatttgccagaggctgggggcccggaagtcacgtacgagccacccccgacagcccccaccggagggggaggatctgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - protein, 357 aa, Homo sapiens
MEFGLSWVFLVALLRGVQCETRYCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPTGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - DNA, 1014 bp, Homo sapiens
  CDS (1)..(1014)
gagacacggtactgcatctactacaacgccaactgggagctggagcgcaccaaccagaccggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgagggcaacttctgcaacgagcgcttcactcatttgccagaggctgggggcccggaagtcacgtacgagccacccccgacagcccccaccggagggggaggatctgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 338 aa, Homo sapiens
ETRYCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPTGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 110 aa, Homo sapiens
ETRWCIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 20 - DNA, 1014 bp, Homo sapiens
  CDS (1)..(1014)
gagacacggtggtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgagggcaacttctgcaacgagcgcttcactcatttgccagaggctgggggcccggaagtcacgtacgagccacccccgacagcccccaccggaggaggaggatctgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 21 - protein, 338 aa, Homo sapiens
ETRWCIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 217 aa, Homo sapiens
APELLGGPSVFLFPPKPKDILMISRTPEVTCVVVDVSHEDPEVKFNWYVGGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 5 aa, Artificial Sequence
  Description of Artificial Sequence Synthetic Linker peptide
GGGGS

SEQ ID NO: 26 - DNA, 36 bp, Artificial Sequence
  Description of Artificial Sequence Synthetic Hinge linker peptide
ggagggggaggatctgtcgagtgcccaccgtgccca

SEQ ID NO: 27 - protein, 12 aa, Artificial Sequence
  Description of Artificial Sequence Synthetic Hinge linker peptide
GGGGSVECPPCP

SEQ ID NO: 28 - protein, 12 aa, Homo sapiens
ERKCCVECPPCP

SEQ ID NO: 29 - protein, 15 aa, Homo sapiens
EPKSCDKTHTCPPCP

SEQ ID NO: 30 - protein, 12 aa, Homo sapiens
ESKTGPPCPSCP

SEQ ID NO: 31 - protein, 18 aa, Homo sapiens
MTAPWVALALLWGSLWPG

SEQ ID NO: 32 - protein, 18 aa, Homo sapiens
MTAPWVALALLWGSLCAG

SEQ ID NO: 33 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 34 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 35 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 36 - protein, 217 aa, Homo sapiens; identical to SEQ ID NO: 23

SEQ ID NO: 37 - DNA, 48 bp, Artificial Sequence
  Description of Artificial Sequence Synthetic Hinge linker peptide
ggagggggaggatctgagcgcaaatgttgtgtcgagtgcccaccgtgc

SEQ ID NO: 38 - protein, 16 aa, Artificial Sequence
  Description of Artificial Sequence Synthetic Hinge linker peptide
GGGGSERKCCVECPPC

SEQ ID NO: 39 - DNA, 42 bp, Artificial
  Hinge linker
ggagggggaggatctggtggaggtggttcaggtccaccgtgc

SEQ ID NO: 40 - protein, 14 aa, Artificial Sequence
  Description of Artificial Sequence Synthetic Hinge linker peptide
GGGGSGGGGSGPPC

SEQ ID NO: 41 - DNA, 42 bp, Artificial
  Hinge linker
ggagggggaggatctggtggaggtggttcaggtccaccggga

SEQ ID NO: 42 - protein, 14 aa, Artificial Sequence
  Description of Artificial Sequence Synthetic Hinge linker peptide
GGGGSGGGGSGPPG

SEQ ID NO: 43 - DNA, 54 bp, Artificial
  Hinge linker
ggagggggaggatctgagcgcaaatgtccaccttgtgtcgagtgcccaccgtgc

SEQ ID NO: 44 - protein, 18 aa, Artificial Sequence
  Description of Artificial Sequence Synthetic Hinge linker peptide
GGGGSERKCPPCVECPPC

SEQ ID NO: 45 - protein, 14 aa, Artificial Sequence
  Description of Artificial Sequence Synthetic Hinge linker peptide
GPASGGPASGPPCP

SEQ ID NO: 46 - protein, 21 aa, Artificial Sequence
  Description of Artificial Sequence Synthetic Hinge linker peptide
GPASGGPASGCPPCVECPPCP

SEQ ID NO: 47 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 16 aa, Artificial Sequence
  Description of Artificial Sequence Synthetic Hinge linker peptide
GGGGSVDKTHTCPPCP

SEQ ID NO: 49 - protein, 16 aa, Artificial Sequence
  Description of Artificial Sequence Synthetic Hinge linker peptide
GGGGSVDKTHTGPPCP

SEQ ID NO: 50 - protein, 21 aa, Artificial Sequence
  Description of Artificial Sequence Synthetic Hinge linker peptide
GGGGSGGGGSVDKTHTGPPCP

SEQ ID NO: 51 - DNA, 2175 bp, Homo sapiens
agtacagtataaaacttcacagtgccaataccatgaagaggagctcagacagctcttaccacatgatacaagagccggctggtggaagagtggggaccagaaagagaatttgctgaagaggagaaggaaaaaaaaaacaccaaaaaaaaaaataaaaaaatccacacacacaaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaacaaaaaaaaaaacaaaaaaaaacaaaagtaaattaaaaacaaaacctgatgaaacagatgaaggaagatgtggaaaaaatccttagccagggctcagagatgaagcagtgaaagagacaggaattgggagggaaagggagaatggtgtaccctttatttcttctgaaatcacactgatgacatcagttgtttaaacggggtattgtcctttccccccttgaggttcccttgtgagccttgaatcaaccaatctagtctgcagtagtgtggactagaacaacccaaatagcatctagaaagccatgagtttgaaagggcccatcacaggcactttcctacccaattacccaggtcataaggtatgtctgtgtgacacttatctctgtgtatatcagcatacacacacacacacacacacacacacacacacacaggcatttccacacattacatatatacacatactggtaaaagaacaatcgtgtgcaggtggtcacacttcctttttctgtaccacttttgcaacaaaacaa

SEQ ID NO: 52 - DNA, 11373 bp, Homo sapiens
gtgcgcggggcggcgccgcggaacatgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatctaagcccaggacatgagtgtctgtccagactcagtggatctgaagaaaaaaggaaaaaaagttgtgttttgttttggaaatcccataaaaccaacaaacacataaaatgcagctgctattttaccttgactttttattattattattataattattataattattattattaatattattttttggattggatcagtttttaccagcatattgctctactgtatcacaaacagcggacacgtcagcaggcgttgaggtgctgagctgtggatgcagaaccagcgccatgctgaagagcctcagccacctcctgtcctttgggattcgtttttcccgctttctctttgtttgtcgtctcagaatctgtgacacaaagaaacccatctcctgtcttaggaaacctaatgctgcaaactctacctagaggaacctttgaagactgttacataagaacataccttcctcagaagaggagtttcctctgccctctgcccttctcccctgcctccctccctcccctccttttattttgttttagtgagcttaagaaacagcagatgtgtctttcacggatctaacgggtgttgtcctgatcgagaaaaaaactgggatgagaatggtttggactggagttggaaggggaggacggtactgggggtagggtttggaacagagctacactggactcgggcacattcggagcagcatcctttagtatggaggctacttctcaggtaaccaggaattgaggggaaggaccttgtggaggccgagcattaacagcaagagcggggtttggagaaagtctgagattgggtgcagccctgacttacctgctggccctgaccagtttcttttcactaacttggccttgggcataggatgaaacattttttctgccctaattttaaaactaggtgagggtagaatcatcacaggttaggaatacattcttcataagacacgatgctgtaaatacccttaatggacgaaaagttgaaatacttttgtttcctcttggagcagttcagggaaatgcccacaggggattgtcctgcacagatagggcaagaggatttcctgggtggagtctgccaaggcctgcctcgctggggaccccagagtcctgcacctctggttccgccccaggtggtgacattactgtccccgttctgtggctcgtggacaagactttctccagaccccttaaagtggtacatattctaaaaaactgtttttctattatgccataaccttgctctagtcagtgaatgttcctaatgctgctgtttcaacatttgaattctttttaatttatgaaacatgctaaattttttttttcaaacaaaacacacacatccacatatacacatgcttcgctatgtggcttccaaggtttaaattttgaaaagtaaaagaattaaaacttcacgaccacagatcacctcaaaccagaaatacctcagaattttctacttatgtaaggtttattatatattttgttagttgtgttgtcttgtagtaagtatattttaatgtaagttggcttttgtgacaaggaagtttaaaagaaatagagaaaaagaaaaaagtttgcatcttctagggagtgctaccatttttgtttgataacgcccccttgtaaataattgtcatcaactgtaggttggctgtctgggccaagtctgggcatttatcagtcttgtttgtgaaggcttttccttctggtttctttagatcattttatttaaaaacagtgcatctcttcatcgtgagggtaggcaaggcgggggccgtggggagaggttgacctgggtgagaactgaagaggccgcctcctcttgggttgtttggagcttcacatgtaattcacatgtaacatgtaacttgatcggtcagtgttcagaatgacaagtaaccccgcttaaacttggtagaaggatggcccttagacctgaatggggtgattttacttgggatttaacttcttcagcaaattaacagcaacgttggaagagatctgtggcgcctctgtgaagcacaccgtgactcaggccagtcttttagtgcagcgtgtctgggagtgaagggttttgcccttgctggtcttggagtccacagtgtgaggggcactgcacatgcctgggcatctacctagtgtgctatgttcagtgtctggggcttactgccccggggtcctttcctctgggtgttggggcacagggtgctatgggaggcccatttgcttccctctcggagctcagtttttgcttcatgggtcaaaatgtgggctggccaagtggttacaggaacagggtttcggtaagctatgttgtctttttttttttttttttttttttttttaatggtttgattttgtgctgtggtattttttttcccttagaataatttttaatggcaaaacaggccttacagcagttgcttttctttaccatttatttctttaagaagctttaaaatatttattgaaaagtgccatatctaatttctttagctttcgcctcaggcagtgcaggcatctttacttttcatcctcagaagaaacaaacgactaacaaatgtagcaaatttactgcaggaatagttaggtcatgatactacctgaacactaaaccccagcctctttgtttggttttagttcctctgggtggtttttcttttgtgtgctggcttgattcttgtgagaagttttgacctggccaagggagggttgagccatggttctggtgtgggactttgcggtcaagacacagtacagacaggtcaggcctgcgtgccttttctctgggtggcctccccgttaggcccaccgtacgctcagccactatagtgtccctgtggggccttgccatcagattgtgtgtcaggagatggtacctttttggtgtggctggggaggagtgtggtccatgccagttctttgggcttcaggccactcttcccctcatgctgtggtgtaaagtgcacccatcaggtggtatatctggttctgatggcaagaagaaggtgggggatctccttatagggcatgggtctaggagcacagatgggccttttgccccgggtaaatgcttgtctgtttgctgtcatgtgttctttgaggagtgagccatctcgagccctgctttgaatttactgggtcatagagcctctgcctgtgctcttttccataatgacttcatgtgacatgcacttttggtgggctcagataattggtttctttttgtttttgacctcaggctctgtggcagactggggaaaatggggcctggcatcattttccctgtcaatgggaggggctgttccatgcagggtgggaggggaccaagttagcagagagtagccaaggatccttgcttcttcctttctagtgtgctgtcatccaagcaggctcctggctgtagggatgggccttggggaagaatcttctttgaaagcatctatgataactgagaagtcatccctagttggagaaatccagtaatgagcagaaggaggaagcaagtgaggacagaggccattgtattacagtgtcacgcagagggccctcaatgatggggcattggggaaggctgtagacatagtcatcagaacatcctggcctggcataagctgggttttctcctgggaccattggtcctcagcaggagttctttgcatgagttgctcaggggcaagggctgcaagtgggctgtgcttaggagaaagtgacacctggcagtgagggaagatggtgagcattattagcctttgttgtccagcatggccttcttgtcctgtctgctctggagaggagcctgtgggaccagtcctgcctggggagggcatacccacacgtgccagctgattctgactctgaatacatcatgtccggacttgggggtgtttctgcagaaaaaggaggttgtttttcagccttgaacatcttcaggaggatagagactcttgctcacatattcttagcaaagggaagggtctctcatctccaggccacagagatagttcttccattgccctaagaggctaggctaaccctcttgacataacttagacagcaaagcacttcatcctgtagttgggctctgtcacctttctcttcagttggccacattctcgtttcctccatcctgctatgctttgtgtgctcgggctgtgtgtggggtttttccctggtggaaggaagcccagctgtgtattgaatgtccttcatgtgttgtgtgtggctcagaaagcctgtcacttggcccctgtgctctgagccgtgagggtggggaggtggctgttccattaaagtgggagtattggatggccctcttgaaactagaattttgccttttttagtatgcagtataaagtttccagcatctattggtaacacaaagatttgctggtttttaaaataatacagtaagcataagtatgtaagtttttagaattggtactagaagttggacagctagttattctcgagaactttatttcactagaaaaatatactaattggaaagcagtttccaggagttaactcagtttaattttcagtctcagttattttagcctgttgagtttttgatggcacacctttggagagatggccacgcctgattcccatttcaggggcatcagaccatacctttttaagaagctccgtgaatctagtcatctacccttcatcctgggcgaacagccaaaaagagaaggggacaaggtgtctttttctccttctcactggggtgacatgaattcttttagttaatggctgtttgcaaattctaaactaatgaaatacttagcagctaacatgttcaatctagtaatgatgagtttaaatctcaattgacagtaatgttttagataaacaggcccagtaattcagttgatgaactgtatatcttctcagtctagatttgtaaatgtttaatgaattcagggttataagcatagttctttaagtaagattccagatagttgatttgcaaccagcagtctacctatgaatgtatcccaaacctttagaagattggaaaagatttttgaaataatgatttagttttgtaggaaaaacacccccttgaaaattaattcggttgacccagtaacattttttaaaacaattggtggctccaaaaggcctgccaacaaagaaaagtccaaattatctagtgggacattttgaatgttttatgtttattttgggtccactgtaaactttggttcaaaaaagaatttgaatttaaagaatttaccattatttaaattattaccaagtttttacattttcatgatggtattttccaggtatgaatgaaacatgactttttgattgtggtacttcctgtatcccctgtagtgccaaaaccagtgatactttatttgctcctatggcagctcatagaggtaaccgaagtgatttttcctcagtaattgaaacacatattctctaaatgccaatgtgtggtgatgggccctgcactgccttcatttctctagggcagtgtctttggattgtctagggcctaggtaattctgagaactactgtaaaccaaccacagggcactaaagcaatgtacacaccactctttgtgtgtatggaaggggttatataaacctgggctatgctggacatctacagaagagtattacattcacttgcaaagtttacatttttgagctcacagttatgaaaaatatgacccacaagtttttcaggcaggtgaggatgggtcttcttgcaaatgcatgagttctgtcttgagtcctgggaacttctctgttggttgagtgtgggctcattccctgactctcctaatcatgtttgcgtcagaatgttagcattgtaaataaaagaataggttgtataatagatacacaacacttgaaactttactttaaaaaaatcgatagttctacatatatatttagttatatcacttgacagatttcttctacacagtgtggagattgttttataccacagattatttttataaagttagtgaatttgaatgattttgtaatcagagctaatgagctttacctttcaagagaaacgtacactggagcatgagtggtgtggaacttttacttagtgtttatatggattcttgtgatacactggcagactggagtcaatttgcgggtcttttttggccaaaactccacttgtggttgtgtaggacagtgatattcagctcagcttcttgtggattgggaggagagagggcctgcaatgtgttttacattggtgcttcctcctgagatttctgttgaacaaagggttctgaggtcaaaaattagtttgtaagcctttgccataggacatagtcatgtgagagtgtttgggggaacagaaattgtataggggtgcctattggggtgggatgggactcgaataagattcaggtacaaaaactttgaaatgagaatctggtggtttgagtaatccaccagactgaattatctaagatcacattatccaggttggggggcagaattacccagttaagtaattgttcagaaaagtggggagggtggcatgtggatgcagtgatccaattaaatggagagctgccaggcacattttgtcctctctggtcagtgagaatggttgggttggctcgctgcttcaatctgtggaatcagccaggagcccagtgaggaagctcagaaccccagtaacagcagagcatctttcagatagctccagagttttcctgcttttctgaggaagctcagcatcactgccacaatacggaaagtggtcttcattttagcctatttatttttaggcagagagtggatggttatttgtgtgggacttttggtggcgatatataatgaataattaagttaatttctggtatgcataatggccagtcctgaggcccagctgaagacctgtcccccagaccctgcccgctggcttcaggctgctgcttctagacagaggtgcactggacgggatagttttatcaagagaatccctaatgtgtcattttaaaccagctgtgctttttattcattctggttgagcgtataggtttacactttaccctttttatacttggaataaatttagttccagcagatctagtagcactccagaaaccaaccccatctgttccccataaaaagaacattttctctgctctccagccacgtgtcttggaatgtaattctgttgtgcctttgtttttatcactctcttcgccccaaaagcaactgctgtaagcttttttctacttgtcttttctagtccccaacctctacctttttcctttttcccagccctaatttctggatgcacttctgtgatccaggtattttaagaaccagttacctcagacctcatgttgaacagtgtcgccatctgggtcctcttgatactgcagacttttaacgtacacatgcaggaaccctgctgagcgtgggcacttgttttaaagcaaaactcttcccaaggactgaagaaagggcttctggcaagctcgtcatggcattgtggtgggatgggtctagagtgtcatctgaatggtgcttcctgtgttcctctttgaattctgccattttcagtattcttgtgtgtctgaataggcaaagcgatttaattggctggtcttgcacgcaaattagttccaaagataagctctttgtaacacatttccagtcgctaatgctcaaatgtagaacattcctttaaatggcaggataaaaaacccactatccaccatagtgcattttgggaagatgtctgtagcatatgttgctgtgaaattaggccttgtgggatatggctgtttgtcattttgatgtattttaaataaatatatatattttttaaagagccttttttaccagttcaaaaagtttaattaaccagcagtcaccgcatctgaatttttgtctctggggcatagatggcagaccaagattaaaagtggtaactcagctatacgagcatgggctaccttcctgggctctcctgcagtcctgtagacctgctgttccgcagaccatgggacacaaggtcagtgtgttcccagtgagggtcccaagtcagtcatcttaagtgtttgttctctgccccattcagtggactgttgacttcagtccctgcaagtgctttagcccgagtggggttttctcagagcactgccacgagttaagtgtgtgtttagccaaataatttctccgtaagggaaaaatgcagtcacccaaattttaccaacaatgacagagatgagagtagaaaagattaggcaacatctgagttttaacttgaaaagtgtccaagtcatcatgaaaggccgactgggagcaagtgattattagagattcttcaggagacctcatctgaaaatgttaagactgccagtgagggaaggaattgttaaaatgccagcggcttttttttcctctttttttctgtaattctgtaaaaatgcagagaaagttgagtggtacttcagaattgagggagagggttaccgcagagtagaaatatatttctagatttcagttccacaccacaaatccacaacaatgccatttttcaactgtacaaaaatctgcttatgaactggacatgatcttaatggtagtgtcaaaggccaagtttttcacctgttaatatttttccacatttgtccttgaatctgaataactttatacagtactgtaaatttaacttacatcgagtttgttgtcaattcttatgaaaagagctttctgcatgtaacacatacggttaaagaacacagcaaaggacaaaatttgcaggaacagttttggaaccaacagaaaatgtcaccttttatttgccatcttatatatatctatcagttttaccagctacttctaaatttgtacattatttgtaagggaaagaaggaaaaccctaagacttgtctaacttagtggagaatgtgtgtgttgggcttaggatggatagctaagtcttattgagctgtgttacctaacttgtatataaaaattgtaattaaaagtttgggttcacctgtttctcacagtttaaaatgatgagtaattgcaaactctggaaatgtgactagtatatgatttaaggctgtagaagcaaggaagctctttcaagtgctaaaactaaagacttctagtttttggctcaaataagtactgtttgtataccaggatatgtgagatgtaaatgtagtaggtcacttttcacccttgtagctataaaataaaaattttgtagaacagaaatagcttgtactactgaattaacaaaagttatactaaagtatcatgtttaaaaaaaatatatatatatatacagagttaagcttgttgctgttaccctgtctggatttgaaaagtgtgctgatttatatatatatattacacacacacacacacacacacacacacacacacacacacacacacacacacacacacacacacacacacacatacacctaaaatggcctaaagcagacatccatgtaattacagttgcaaaatgaaaacattttggaaagaacattgtatcatagttcattcatttgcagtggatctttgttcctttttactgtggtaattttagaaatgagtgtcaagtttgaaattagatctgctaagttggggttttgctgcttgaactctgcactgggtcctcaaataaaccgatgtgaatgtagttttttccccctgtgtgaagaagcagttacaccccaacaataggaggaaaaatctagaactatttcaagttttatctttttgtatatgaaaataaaataataataaaacaa

SEQ ID NO: 53 - protein, 11 aa, Artificial Sequence
  Description of Artificial Sequence Synthetic Hinge linker peptide
GGGGSVECPPC